SEQUENCE LISTING (13 sequences)

SEQ ID NO: 1 - RNA, 18 nt, Artificial Sequence
  siRNA
guaaucgcaaguucaaau

SEQ ID NO: 2 - RNA, 19 nt, Artificial Sequence
  siRNA
gaacagacaggcuuacuua

SEQ ID NO: 3 - DNA, 30 bp, Artificial Sequence
  Primer
ctgcaggaattcgaacgctgacgtcatcaa

SEQ ID NO: 4 - DNA, 62 bp, Artificial Sequence
  Primer
aaatctcttgaatttaacaatacccagctccggggatctgtggtctcatacagaacttataa

SEQ ID NO: 5 - DNA, 59 bp, Artificial Sequence
  Primer
ccatcgataagctttttttccaaaaaaggagctgggtattgttaaatctcttgaattta

SEQ ID NO: 6 - DNA, 21 bp, Artificial Sequence
  Primer
tgttaccagggaggagcagtg

SEQ ID NO: 7 - DNA, 21 bp, Artificial Sequence
  Primer
tcttgcccttcctttctgtca

SEQ ID NO: 8 - DNA, 27 bp, Artificial Sequence
  Primer
caggactctaatccagagtttaccttc

SEQ ID NO: 9 - DNA, 16 bp, Artificial Sequence
  Primer
gggatggctgccagca

SEQ ID NO: 10 - DNA, 22 bp, Artificial Sequence
  Primer
agcagccctacgaccaggccca

SEQ ID NO: 11 - DNA, 21 bp, Artificial Sequence
  Primer
gccaaactacagcgaactgga

SEQ ID NO: 12 - DNA, 21 bp, Artificial Sequence
  Primer
tgtggtatgacaggcatggag

SEQ ID NO: 13 - DNA, 31 bp, Artificial Sequence
  Primer
cacatacagtgattatttccccgtatctcta

(19) United States
(12) Patent Application Publication
(10) Pub. No.: US 2013/0324438 A1
(43) Pub. Date: Dec. 5, 2013

(54) METAGENE EXPRESSION SIGNATURE FOR PROGNOSIS OF BREAST CANCER PATIENTS

(71) Applicants: Geert Berx, Linter-Wommersom (BE); Eric Raspe, Mouscron (BE)

(72) Inventors: Geert Berx, Linter-Wommersom (BE); Eric Raspe, Mouscron (BE)

(21) Appl. No.: 13/882,120

(22) PCT Filed: Oct. 31, 2011
(86) PCT No.: PCT/EP11/69161

§ 371 Date: Jul. 10, 2013

(30) Foreign Application Priority Data
Oct. 29, 2010 (GB) 1018312.7

(51) Int. Cl. C12Q 1/68

(52) U.S. Cl. CPC C12Q 1/6886
USPC 506/10; 506/16; 506/17

(57) ABSTRACT

Described are gene expression signatures able to distinguish individuals having or suspected to have breast cancer with good clinical prognosis from individuals with poor clinical prognosis, based on ZEB2 transcriptional activity. Further described are kits and assays related to the prognosis and/or the change in prognosis of the individuals suffering from breast cancer.

### CROSS-REFERENCE TO RELATED APPLICATIONS

This application is a national phase entry under 35 U.S.C. §371 of International Patent Application PCT/EP2011/069161, filed Oct. 31, 2011, designating the United States of America and published in English as International Patent Publication WO2012/056047 A1 on May 3, 2012, which claims the benefit under Article 8 of the Patent Cooperation Treaty to United Kingdom Application Serial No. 1018312.7, filed Oct. 29, 2010.

### TECHNICAL FIELD

The disclosure relates to the field of biotechnology generally, and particularly genetic marker genes useful in the diagnosis, prognosis, and/or prediction of cancer. More particularly, it relates to gene expression signatures able to distinguish individuals having or suspected to have breast cancer with a good clinical prognosis from individuals with a poor clinical prognosis. Such genetic profiling will also provide guidance for patient treatment and is useful to monitor disease outcome. Further provided are kits and assays related to the prognosis of the individuals suffering from breast cancer.

### BACKGROUND

It is rare for a cancer patient to die due to the local effects of their primary tumor. Rather, it is the metastatic spread of tumor cells that is ultimately responsible for the vast majority of cancer morbidity and deaths. Understanding the cell and molecular biology of invasion and metastasis and the genetic changes that drive these processes represents one of the last great frontiers of exploratory cancer research. Therapies directed against metastatic cells hold the promise of clearing the body of tumor cells and curing the patient. Currently, only a handful of treatments are available for specific types of cancer, and these provide no guarantee of success. In order to be most effective, these treatments require not only an early detection of the malignancy, but a reliable assessment of the severity of the malignancy.

The mechanisms leading to the metastatic dissemination of tumor cells appear to be similar for many different types of cancer and are associated with multiple cellular processes. These include the transition of tumor cells from an epithelial, adhesive phenotype to cells with mesenchymal morphology and migratory and invasive capabilities, invasion into surrounding tissue, intravasation into blood or lymphatic vessels, survival and dissemination through the blood or lymphatic circulation, colonization of distant organs by adhesion to the vessel wall, extravasation and invasion into distant organ parenchyma, and finally metastatic outgrowth in the distant organ (Sleeman, 2000). Thus, metastasis is a highly complex problem with many facets.

Breast cancer, the most common cancer among women (Jemal et al. 2007), is a heterogeneous disease in terms of tumor histology, clinical presentation and response to therapy. Global gene expression profiling of breast tumors allowed molecular classification of breast cancers into five distinct intrinsic subtypes. These are (i) generally ER-positive luminal A, (ii) generally ER-positive luminal B, (iii) ER-negative normal-like (expressing epithelial markers, such as, E-cadherin and cytokeratins 8 and 18), (iv) HER2+ (overexpressing ERBB2 oncogene), and (v) basal-like (tumors expressing markers of the myoepithelium of the normal mammary gland, such as, basal cytokeratins CK5/6, CK14, p63 and epidermal growth factor receptor) (Perou et al. 2000; Sorlie et al. 2001; Sotiriou et al. 2006). This molecular taxonomy is clinically significant since patients with basal-like tumors have the worst overall survival, reflected by the abundance of triple negative tumors (ER-negative, PR-negative and ERBB2-negative), and since patients with tumors of the HER2+ subtype also have a reduced survival. Among the luminal subtype of tumors, the luminal B tumors have a less favorable outcome than luminal A tumors. Several lines of evidence indicate that epithelial-to-mesenchymal transition (EMT) likely occurs in the genetic context of the basal breast cancers and suggest that this tendency to mesenchymal transition might be related to the aggressiveness and the characteristic spread of these tumors (Sarrio et al. 2008). During EMT, epithelial cells lose their epithelial features and acquire a fibroblast-like morphology, with cytoskeletal reorganization, loss of cell-cell junctions, upregulation of mesenchymal markers, and enhancement of motility, invasiveness and metastatic capabilities (Thiery et al. 2009). One key feature of EMT is the downregulation of E-cadherin, a cell-cell adhesion molecule present in the plasma membrane of normal epithelial cells and a gatekeeper of epithelial differentiation. A series of EMT-inducing transcription factors, notably Snail, E47, Slug, ZEB1/deltaEF1, ZEB2/SIP1, Twist, Gooscecoid and FOXC2 plays a key role in EMT at the transcriptional level. It has been proposed that these transcription factors are induced by a series of EMT-inducing signals emanating from the tumor-associated stroma (Berx et al. 2007). The EMT-inducing transcription factors are misexpressed in various types of human carcinomas, including breast cancer (Comijn et al. 2001; Elloul et al. 2005; Rodenhiser et al. 2008).

While mechanism of tumorigenesis for most breast carcinomas is largely unknown, there are genetic factors that can predispose some women to developing breast cancer, e.g., BRCA1, BRCA2 (Miki et al. 1994), c-erb-2 (HER2) and p53 (Beenken et al. 2001). Besides these, non-genetic factors also have a significant effect on the etiology of the disease. Regardless of the cancer's origin, breast cancer morbidity and mortality increases significantly if it is not detected early in its progression. Thus, considerable effort has focused on the early detection of cellular transformation and tumor formation in breast tissue.

A marker-based approach to tumor identification and characterization promises improved diagnostic and prognostic reliability. Typically, the diagnosis of breast cancer requires histopathological proof of the presence of the tumor, in addition to diagnosis, histopathological examinations also provide information about prognosis and selection of treatment regimens. Prognosis may also be established based upon clinical parameters, such as, tumor size, tumor grade, the age of the patient, and lymph node metastasis.

In clinical practice, accurate diagnosis of various subtypes of breast cancer is important because treatment options, prognosis, and the likelihood of therapeutic response all vary broadly depending on the diagnosis. Accurate prognosis, or determination of distant metastasis-free survival could allow the oncologist to tailor the administration of adjuvant chemotherapy, with women having poorer prognoses being given the most aggressive treatment. Furthermore, accurate prediction of poor prognosis would greatly impact clinical trials for new breast cancer therapies, because potential studied patients could then be stratified according to prognosis. Trials could then be limited to patients having poor prognosis, in turn making it easier to discern if an experimental therapy is efficacious.

Accepted prognostic and predictive factors in breast cancer include age, tumor size, axillary lymph node status, histological tumor type, pathological grade and hormone receptor status. A large number of other factors have been investigated for their potential to predict disease outcome, but these have in general only limited predictive power (Isaacs et al. (2001).

Gene expression profiling has been used to develop genomic tests that may provide better predictions of clinical outcome than the traditional clinical and pathological standards. For example, a collection of 70 markers was identified for breast cancer that could classify an individual as having a good prognosis or poor prognosis (Van't Veer et al. 2002).

Although the power of gene expression analysis in the identification of prognosis-relevant genes has been demonstrated, there still exists a need in the art for the availability of reliable prognosis-relevant markers for detecting the metastasis potentiality of a breast cancer tumor, for both medical treatment and medical survey purposes.

### SUMMARY OF THE DISCLOSURE

The disclosure relates to methods of and associated means for finding a gene expression signature (or a gene expression profile, which is equivalent in wording) that predicts disease relapse and may be added to current clinico-pathological risk assessment to assist physicians in making treatment decisions. The role of the transcription factor ZEB2/SIP1 in breast cancer and in particular its contribution to malignant progression was examined. ZEB2/SIP1 is important for the invasive and metastatic behavior of basal breast cancer cells. Surprisingly, it was shown that ZEB2-associated gene expression (i.e., ZEB2 metagene) is predictive for the outcome of breast cancer patients.

Thus, provided is a method of prognosing an individual suffering from or suspected of suffering from breast cancer, the method comprising the steps of:

(i) providing a sample from the individual comprising breast cancer cells or suspected of comprising breast cancer cells;

(ii) establishing a gene expression profile by quantifying, in the sample, the expression level of a plurality of genes comprising any combination of at least 8 genes from Table 1;

(iii) comparing the gene expression profile with a reference gene expression profile; and

(iv) classifying the individual as having a good prognosis or a poor prognosis according to the comparison in step (iii).

In a specific embodiment of the above method, the reference gene expression profile is established by quantifying the differential expression level of the corresponding at least 8 genes as quantified in at least two reference samples that differentially express ZEB2. Preferably, a first reference sample endogenously expresses ZEB2 and a second reference sample only differs from the first in that the expression of ZEB2 is knocked-down.

An increasing correlation coefficient between the gene expression profile and the reference gene expression profile indicates a poor prognosis for breast cancer in the subject, and a decreasing correlation coefficient between the gene expression profile and the reference gene expression profile indicates a good prognosis for breast cancer in the individual.

In another specific embodiment, the reference sample is a reference cell line, such as, a breast cell line or a breast cancer cell line. More specifically, the reference cell line is a basal-like breast cancer cell line, such as, a MDAMB231 cell line.

In any of the above methods, the expression level of the at least 8 genes can be quantified by measuring the level of transcription, such as, by using a DNA array or quantitative RT-PCR or multiplex quantitative RT-PCR.

In a particular embodiment, the sensitivity and/or specificity of any of the above methods is at least 80%.

Further, also described is a method for monitoring a change in the prognosis of an individual suffering from or suspected of suffering from breast cancer, the method comprising the steps of:

(i) applying any of the above methods to the individual at one or more successive time points, whereby the prognosis of breast cancer in the individual is determined at the successive time points;

(ii) comparing the prognosis of breast cancer in the individual at the successive time points as determined in (i); and

(iii) finding the presence or absence of a change between the prognosis of breast cancer in the individual at the successive time points as determined in (i).

In particular, the change in prognosis of breast cancer in the individual is monitored in the course of a medical treatment of the subject.

Also provided is a kit for prognosing an individual suffering from or suspected of suffering from breast cancer, characterized in that it comprises the necessary tools for carrying out any of the above methods.

Further provided is an oligonucleotide array or microarray (“(micro)array”) comprising a plurality of probes complementary and hybridizable to nucleotide sequences of any combination of at least 8 genes from Table 1, wherein the plurality of probes is at least 50% of probes on the (micro)array.

In still another aspect of the disclosure, a gene expression profile indicative for a good prognosis or a poor prognosis of an individual suffering from or suspected of suffering from breast cancer comprising a quantified expression level of a plurality of genes comprising any combination of at least 8 genes from Table 1. A reference gene expression profile as defined above is also envisaged here.

Also provided is the use of the above gene expression profile of reference gene expression profile in any of the above methods.

### BRIEF DESCRIPTION OF THE DRAWINGS

FIG. 1: Expression of EMT-inducing transcription factors in MDAMB231. Panel A: We compared the intensity of ZEB2 expression for each cell line in published micro-array studies with the corresponding EPCAM expression values used as marker of epithelial character. ZEB2 expression levels for each cell line common to the three studies were averaged and compared to the corresponding EPCAM expression values. Panel B: Quantitative RT-PCR for ZEB2/SIP1 and EPCAM in different breast cancer cell lines as described in the material and method section. Panel C: Quantitative RT-PCR for ZEB1/δEF1, ZEB2/SIP1, SNAI2 and SNAI1 in MDA-MB-231. Normalized expression levels (average±SD) are compared to the level of SNAI1, which was arbitrarily set at 1. Panel D: Quantitative RT-PCR for ZEB2/SIP1 and ZEB1/δEF1 in MDAMB231 cells stably transduced with empty vector (pLVTH) or vector containing a ZEB2/SIP1-directed short hairpin (shZEB2). Normalized expression levels are compared to the level in control cells, which was arbitrarily set at 1.

FIG. 2: Expression of marker genes in human breast cancer cell lines. Gene expression data from the GSE10890, GSE12777 and GSE16795 studies published in GEO involving at least 20 different breast cell lines were extracted from the corresponding cell files, background-subtracted, normalized and summarized (median polish option) using frozen RMA. The summarized values (in log scale) for each selected probe set for each cell line were converted to a linear scale and normalized by removing the minimal intensity value considered as background and dividing these values by the difference between the maximal and the minimal intensity values. Heatmap was drawn with the heatmap.2 function of the R package gplots, using the average normalized intensity values from the three studies, the Spearman correlation coefficient as distance metric, and the average clustering method.

FIG. 3: Expression of ZEB2/SIP1 in human tumor sample. Expression of ZEB2/SIP1 was monitored by quantitative RT-PCR in breast tumor samples. The ZEB2/SIP1 expression level was compared to that in a panel of representative breast cancer cell lines, including the parental pLVTH- and shZEB2-transduced MDAMB231 cells. Normalized expression levels are compared to the level in the parental MDAMB231 cells arbitrarily set at 1. The average ZEB2/SIP 1 relative expression levels in samples segregated according to their grade, ER and PR status is significantly lower in ER-positive and PR-positive tumors (p=0.0011 and 0.011, respectively).

FIG. 4: Association of the tumor ZEB2 activity index with relapse risk. The ZEB2 activity index was computed and stratified in dichotomic categories defined as whether or not the ZEB2 activity index is above a threshold chosen to obtain the highest logrank Chi-squared value for association with relapse-free survival time or quarters categories defined as the quarter of the range in which the ZEB2 activity index is included. The top panel gives the relapse-free survival probability over time for the merged dataset with data stratified in quarters or the range, while the bottom panels achieve the same for individual studies with dichotomic data. The legends give the number of patients in each group.

FIG. 5: Stability of Cox analysis parameters upon cross-validation. Patients from the pooled data set were randomly distributed 100 times into a training set comprising 75% of the samples (n=1050) and a complementary validation set comprising the remaining samples (n=350). All plots are based on data obtained with the dichotomic ZEB2 activity index values. These index values were built on each training set with either the full list of selected ZEB2 target gene probe sets (ZEB2AI36) or with two subsets of these probe sets. The latter were selected first by removing one by one all probe sets except the ZEB2 probe set from the initial list. Next, a ZEB2 activity index was computed for each of these probe set lists. Then, the list with the highest logrank Chi squared value for association with relapse-free survival time was selected after choosing the optimal cutoff point for the corresponding ZEB2 activity measure. The procedure was repeated until the final list contained five probe sets. ZEB2AI16 corresponds to the optimal list providing the best reproducibility both in cross-validation and in inter-study analysis, regardless of the way the ZEB2 activity index is expressed. ZEB2AI10 provides the best reproducibility in cross-validation only when dichotomic ZEB2 activity index values are considered. To help display the results on a log scale, p-values of 0 were artificially set to 1×10-16. The gene expression values of the ZEB2 probe set were used as reference (ZEB2). Frequencies of occurrence of p-values below 0.05 and of hazard ratios above 1 in the training or validation sets for the ZEB2 probe set and activity indexes are displayed in the lower panel for the training and validation sets, respectively.

Table 1. Common genes down-regulated upon ZEB2 knock-down in MDAMB231 cells. In bold: genes down-regulated more than twofold upon transient knock-down. In italic: probe sets present in ZEB2AI16 list.

Table 2. Characteristics and samples IDs of the cell lines included in the study.

Table 3. References, characteristics and clinical parameters of the breast cancer clinical studies included in the analysis. The number of samples analyzed per parameter is indicated for each study.

Table 4. Association of ZEB2 or ZEB2 activity indexes with hazard of relapse in breast cancer. Influence (hazard ratio and p-value of the logrank test) of the ZEB2 expression level or the ZEB2 activity index computed with the initial 36 probes list or with the optimized list of 16 probes was evaluated by Cox survival analysis using the pooled data or the data or the individual studies as indicated. Note that the GSE12276 and GSE9195 studies have by design unbalanced population distributions according to the question asked (relation between gene expression and metastasis site or resistance to hormone therapy, respectively).

Table 5. Cox survival analysis parameters

Cox survival analysis parameters (time averaged baseline hazard (baseline hazard), hazard ratio, and logrank test p-value) as determined for each study using the Survival R package. The illustrated parameters are associated with each selected probe or with the Spearman correlation coefficient corresponding to the initial list of 36 probes sets (ZEB2AI36; all probe sets) or to the core list of 16 probe sets defined by a leave-one-out approach (ZEB2AI16; first 16 probe sets). In the hazard ratio (H.R.) columns, non-italic and italic data, respectively, are associated with increased or decreased hazard. In the p-value columns, italic and non-italic data correspond to significant or non-significant data at the 0.05 level.

Table 6. Cox survival analysis parameters.

Cox survival analysis parameters determined using the Survival R package. The analysis was based on the Spearman correlation coefficients computed with the full list of probes (ZEB2AI36) or the core list of 16 probe sets defined by a leave-one-out approach (ZEB2AI16). The values were obtained by considering unstratified Spearman correlation coefficients and Spearman correlation coefficients stratified on the basis of quartiles or dichotomic threshold values of the merged dataset. The following parameters are indicated: hazard ratio, logrank test p value, lower 0.95 confidence interval for the hazard ratio, upper 0.95 confidence interval for the hazard ratio, and the p-value for the test of the proportional-hazards assumption.

Table 7. List of probe sets used to compute the optimal ZEB2 activity index.

Table 8. List of reference breast cell lines.

Table 9. Reference vector used to compute the ZEB2 activity index. RNA was extracted from the parental pLVTH- and shZEB2-transduced MDAMB231 cells and hybridized to Affymetrix HG-U133plus2 microarrays. The gene expression data corresponding to the indicated probe sets were extracted from the corresponding cell files, background-subtracted, normalized and summarized (median polish option) using frozen RMA. The summarized values (in log scale) for each indicated probe set for each cell line were converted to a linear scale. The differences between the expression levels of the indicated probe sets in the MDAMB231 cells transduced with the empty vector pLVTH (noted WT) or the vector allowing the expression of the short hairpin RNA against ZEB2 (noted ZEB2KO) are reported.

Table 10. Distribution of the patients among the subsets used to define the optimal probe set list.

Table 11. Criteria for inclusion of patients in the different patient sets.

Table 12. Parameters used to select the probe set lists.

R=raw ZEB2 activity index values, Q=ZEB2 activity index stratified in quarters categories, T=ZEB2 activity index stratified in dichotomic categories (between brackets: lower and upper increment values used to define the threshold in order to avoid that one of the categories contains all the samples). In the case of the raw ZEB2 activity index values, the hazard ratio (H.R.) or the scaled hazard ratio (noun. H.R.) are used as optimization variable.

Table 13. Cox analysis and accuracy performance of the various probe set lists according to the ways the ZEB2 activity index is expressed.

For each method of expressing the ZEB2 activity index, the first column reports the counts of individual studies with a significantly increased hazard of relapse associated with the ZEB2 activity index. The second column reports the counts of patient sets with a significantly increased hazard of relapse associated with the ZEB2 activity index in 100% of the training set in the cross-validation analysis. The third column reports the counts of patient sets with a significantly increased hazard of relapse associated with the ZEB2 activity index in at least 85% of the validation sets in the cross-validation analysis. The fourth column reports the counts of patient sets with a logrank p-value above 0.05 (non-significant association of the ZEB2 activity index with relapse hazard indicated in orange). For the ZEB2 activity index expressed as dichotomic categories, the first column reports counts of patient sets with a sensitivity above 0.3 when the specificity is above 0.85. The second column reports the average sensitivity calculated on the seven patient sets, and the third column reports the corresponding average specificity. The last column reports the counts of patient sets with a p-value below 0.05 according to Fisher's exact test. The values of List3P6 correspond to the selected list values of the core list of 16 probe sets (ZEB2AI16).

### DETAILED DESCRIPTION OF THE DISCLOSURE

The disclosure will be described with respect to particular embodiments and with reference to certain drawings but it is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g., “a” or “an,” “the,” this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention, described herein, are capable of operation in other sequences than described or illustrated herein.

Unless otherwise defined herein, scientific and technical terms and phrases used in connection with the disclosure shall have the meanings that are commonly understood by those of ordinary skill in the art. Further, unless otherwise required by context, singular terms shall include the plural and plural terms shall include the singular. Generally, nomenclatures used in connection with, and techniques of molecular and cellular biology, genetics and protein and nucleic acid chemistry and hybridization, described herein, are those well known and commonly used in the art.

The disclosure provides gene expression profiles for the identification of conditions or indications associated with cancer, in particular breast cancer. Where the gene expression profile correlates with a certain condition, the gene expression profile is a marker for that condition. Generally, the gene expression profiles of the disclosure were identified by determining sets of co-regulated genes or genes involved in common signaling pathways having expression patterns that correlate with the conditions or indications. In particular, gene expression profiles associated with the transcriptional activity of EMT inducers were identified that have a predictive value for breast cancer patient survival probability. More particularly, the disclosure identified ZEB2-associated gene expression as being predictive for the outcome or prognosis (good or poor) of breast cancer patients.

As previously mentioned herein, prior art studies disclosed several gene signatures containing genes potentially involved in metastatic processes and/or markers of distant relapses. However, these prior art studies tackled overall relapse problems. As there are multiple types of metastases and potentially multiple distinct pathological processes leading to metastasis, these prior art studies suffered for lack of accuracy. In view of improving the accuracy of metastasis-specific markers for breast cancer, the inventors have designed an original method for selecting highly reliable prognostic biomarkers based on the finding that EMT inducers, such as, ZEB2, are key factors in setting the malignancy of breast cancer tumors. It was surprisingly found that ZEB2-associated gene expression (ZEB2 metagene) is predictive for the outcome of breast cancer patients in most interpretable clinical studies published so far, and not the expression of the genes taken individually (including ZEB2 itself). Hence, reducing ZEB2 transcriptional activity in the malignant compartment of the tumor can be useful for preventing or curing breast cancer relapse. As tumors with a gene expression profile closest to the profile acquired after ZEB2 knock-down are the most likely to relapse, targeting ZEB2 activity with small molecules that interact directly with ZEB2 or affect signaling pathways or enzymatic activities modulating ZEB2 activity or sub-cellular location can significantly improve our therapeutic arsenal. In this regard, it was shown that reducing the ZEB2 activity through ZEB2 knock-down blocks in vitro two- and three-dimension MDAMB231 cell migration, lung colonization after tail vein injection, anchorage-independent growth and growth of MDAMB231 xenografts (WO2009/106578). Thus, reducing the ZEB2 activity in breast tumor cells by a drug can reduce their aggressiveness and thereby reduce the risk of relapse for the patient treated with that drug. Furthermore, measuring the ZEB2 transcriptional activity by profiling sets of ZEB2 regulated genes could be used to identify patients who would benefit the most from targeted aggressive therapies and to follow the outcome.

Thus, provided is a method of prognosing an individual suffering from or suspected of suffering from breast cancer, the method comprising the steps of:

(i) providing a sample from the individual comprising breast cancer cells or suspected of comprising breast cancer cells;

(ii) establishing a gene expression profile by quantifying in the sample the expression level of a plurality of genes comprising any combination of at least 8 genes from Table 1;

(iii) comparing the gene expression profile with a reference gene expression profile; and

(iv) classifying the individual as having a good prognosis or a poor prognosis according to the comparison in step (iii).

According to specific embodiments, the reference gene expression profile is established by quantifying the differential expression level of the corresponding at least 8 genes as quantified in at least two reference samples that differentially express ZEB2. Preferably, a first reference sample endogenously expresses ZEB2 and a second reference sample only differs from the first in that the expression of ZEB2 is knocked-down.

In a more specific embodiment, provided is a method of prognosing an individual suffering from or suspected of suffering from breast cancer, the method comprising the steps of:

(i) providing a sample from the individual comprising breast cancer cells or suspected to comprise breast cancer cells;

(ii) establishing a gene expression profile by quantifying in the sample the expression level of a plurality of genes comprising any combination of at least 8 genes from Table 1;

(iii) comparing the expression level of the at least 8 genes in the sample with the differential expression level of the corresponding at least 8 genes between at least two reference cell lines, wherein a first reference cell line endogenously expresses ZEB2 and wherein a second reference cell line only differs from the first reference cell line in that the expression of ZEB2 is knocked-down; and

(iv) classifying the individual as having a good prognosis or a poor prognosis according to the comparison in step (iii).

Preferably, the reference sample is a reference cell line, such as, a breast cell line or a breast cancer cell line. More specifically, the reference cell line is a basal-like breast cancer cell line, such as, a MDAMB231 cell line.

In the context of the disclosure, prognosing an individual suffering from or suspected of suffering from breast cancer refers to a prediction of the survival probability of individual having breast cancer or relapse risk, which is related to the invasive or metastatic behavior (i.e., malignant progression) of breast tumor tissue or cells. As used herein, “good prognosis” means a desired outcome. For example, in the context of breast cancer, a good prognosis may be an expectation of no recurrences or metastasis within two, three, four, five years or more of initial diagnosis of breast cancer. “Poor prognosis” means an undesired outcome. For example, in the context of breast cancer, a poor prognosis may be an expectation of a recurrence or metastasis within two, three, four, or five years of initial diagnosis of breast cancer. Poor prognosis of breast cancer may indicate that a tumor is relatively aggressive, while good prognosis may indicate that a tumor is relatively nonaggressive.

As used herein, the term “individual” or “subject” or “patient” typically denotes humans, but may also encompass reference to non-human animals, preferably warm-blooded animals, more preferably mammals, such as, e.g., non-human primates, rodents, canines, felines, equines, ovines, porcines, and the like.

As used herein, a “sample” from an individual suffering from or suspected of suffering from breast cancer means a sample comprising breast cancer cells or suspected to comprise breast cancer cells. The sample may be collected in any clinically acceptable manner, but must be collected such that nucleic acids, are preserved, in particular mRNA or nucleic acids derived therefrom (i.e., cDNA or amplified DNA). A sample may comprise any clinically relevant tissue sample, such as, a tumor biopsy or fine needle aspirate, or a sample of bodily fluid, such as, blood, plasma, serum, lymph, ascitic fluid, cystic fluid, urine or nipple exudate. The sample may be taken from a human, or, in a veterinary context, from non-human animals, such as, ruminants, horses, swine or sheep, or from domestic companion animals, such as, felines and canines. The sample may also be paraffin-embedded tissue sections. It is understood that the breast cancer tissue includes the primary tumor tissue as well as a organ-specific or tissue-specific metastasis tissue.

As stated above, gene expression profiles comprising expression values of genes associated with the transcriptional activity of ZEB2 were identified (i.e., ZEB2 metagene) that may have a predictive value for breast cancer patient survival probability, based on gene expression changes induced upon ZEB2 knock-down in reference breast cell populations. ZEB2 (also known as Smad-interacting protein SIP1) is a transcription factor that belongs to the δEF-1 of ZEB protein family and is known to be a potent EMT inducer (Comijn et al. 2001; Vandewalle et al. 2005). The methods that were used for identifying such prognostic gene expression profiles are further described in the example section and form fully part of the disclosure.

“A gene expression profile” is equivalent in wording as “a gene expression signature” and these wordings are used interchangeably herein. In the context of the disclosure, a “gene expression profile” refers to a profile of expression levels of a plurality of genes wherein the gene expression profile is a prognostic marker for individuals having breast cancer. A gene that appears in a gene expression profile is said to be a member of the gene expression profile. For example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 member genes can be selected from Table 1 for an optimum signature for prognosis of individuals having breast cancer.

As used herein, a “prognostic marker” means a biological marker, which is differentially expressed in breast tumors that generate metastasis, or will generate metastasis, as compared to the expression of the same biological marker in breast tumors that do not generate metastasis, or will not generate metastasis.

In a particular embodiment of the above-described method, a gene expression profile can be determined by quantifying the expression level of a plurality of genes comprising any combination of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 genes from Table 1. More specifically, the plurality of genes can be selected from the group comprising ANK2, ANK3, CADPS2, CASP1, CCND2, COL6A3, CXorf57, EDNRA, EFNB2, ENOX2, GAD1, HES1, IGFBP1, IL7, JAG1, KRT15, LTBP1, MAP3K5, MFAP3L, NDP, OASL, PDE2A, PLA2G4A, PORCN, RGS4, SCG5, SLC22A3, STC1, TBC1D8B, TCN1, THBD, TPK1, VNN1, XK and ZEB2. Preferably, a gene expression profile can be determined by quantifying the expression level of a plurality of genes comprising any combination of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 genes from Table 5. More specifically, the plurality of genes can be selected from the group comprising ANK2, ANK3, CADPS2, CCND2, COL6A3, CXorf57, EDNRA, EFNB2, ENOX2, GAD1, HES1, IGFBP1, IL7, JAG1, KRT15, LTBP1, MAP3K5, MFAP3L, NDP, OASL, PDE2A, PLA2G4A, PORCN, RGS4, SCG5, STC1, TBC1D8B, TCN1, THBD, TPK1, VNN1, XK and ZEB2. In more preferred embodiments, a gene expression profile can be determined by quantifying the expression level of a plurality of genes comprising any combination of at least 2, 3, 4, 5, 6, 7, 8, 9, 10, 11, 12, 13, 14, 15 or 16 genes from Table 7. More specifically, the plurality of genes can be selected from the group comprising ANK2, ANK3, CADPS2, CCND2, COL6A3, CXorf57, HES1, NDP, OASL, PLA2G4A, PORCN, RGS4, SCG5, TPK1, XK and ZEB2. In more preferred embodiments of the above-described method, a gene expression profile can be determined by quantifying the expression level of a plurality of genes comprising each of the following genes: ANK2, ANK3, CADPS2, CCND2, COL6A3, CXorf57, HES1, NDP, OASL, PLA2G4A, PORCN, RGS4, SCG5, TPK1, XK and ZEB2. It is understood that a gene expression profile can be further refined and optimized as presented in the example section. According to a particular preferred embodiment, the gene expression profile is determined by quantifying the expression level of a plurality of genes as described above, further characterized in that at least ZEB2 is comprised within the plurality of genes. Or in other words, that ZEB2 is a member gene of the gene expression profile as defined hereinbefore.

The names of the genetic markers as comprised in the gene expression profile and specified herein correspond to their internationally recognized acronyms that are usable to get access to their complete amino acid and nucleic acid sequences, including their complementary DNA (cDNA) and genomic DNA (gDNA) sequences. The corresponding amino acid and nucleic acid sequences of each of the genes specified herein may be retrieved, on the basis of their acronym names or gene symbols, and/or on the basis on their gene ID, in the GenBank or EMBL sequence databases. All gene symbols and gene IDs listed in the present specification correspond to the GenBank nomenclature. Their DNA (cDNA and gDNA) sequences, as well as their amino acid sequences are thus fully available to the one skilled in the art from the GenBank database, notably at the following website address: worldwide web at ncbi.nlm.nih.gov/. For the purpose of being illustrative, one example of an acronym or gene symbol, as used herein, is “ANK2,” and the corresponding gene ID is “273” (Table 1). The same sequences may also be retrieved from the Hugo Gene Nomenclature Committee (HGCN) database that is available at the following website address: worldwide web at genenames.org/.

The disclosure provides methods of using a gene expression profile to analyze a sample from an individual so as to determine the metastatic potential of an individual's tumor at a molecular level, i.e., to determine a prognosis for the individual from which the sample is obtained. The individual need not actually be having breast cancer. Essentially, the gene expression profile comprising expression levels of sets of genes in the individual, or a sample taken therefrom, is determined and compared to a reference gene expression profile. Based on this comparison, it can be determined if the pattern of expression indicates a good or a poor prognosis. It should be understood that a gene expression profile and a reference gene expression profile are based on the expression levels of corresponding set of genes.

In the context of the disclosure, a “reference gene expression profile” or otherwise a “standard gene expression profile” or “control gene expression profile” refers to a gene expression profile that is determined by quantifying the differential expression of corresponding sets of genes between two reference samples that differentially express ZEB2, preferably wherein a first reference sample endogenously expresses ZEB2 and wherein a second reference sample differs from the first reference sample in that the expression of ZEB2 is either absent or knocked-down. As used herein, a reference sample can be a tumor sample of a breast cancer subtype expressing or not ZEB2 or a breast cell line sample of a subtype expressing or not ZEB2. As used herein, a “reference breast cell line” can be any breast cell line known in the art, including in a non-limiting way the breast cell lines as listed in Table 8. Thus, a reference breast cell line can be a normal breast cell line or a breast cancer cell line. In a particular embodiment, the reference breast cell line without expression of ZEB2 can be the same as that expressing ZEB2 provided that ZEB2 mRNA or protein levels or activity is reduced by any means known to those skilled in the art, such as, siRNA, shRNA or aptamers. In a further particular embodiment, the reference breast cell line is a basal-like breast cancer cell line, such as, MDA-MB-231.

As used herein, “knock-down of ZEB2” or “ZEB2 knock-down” means a reduction of the activity of ZEB2 by at least 70%, preferably by at least 80% or at least 90% or at least 95%, or by 100%. This reduction can be achieved by reducing the expression or the protein level or the activity of ZEB2 by any means known to those skilled in the art, such as, siRNA, shRNA or aptamers.

A non-limiting example of a reference gene expression profile based on the differential expression level of a plurality of genes is provided in Table 9. The person skilled in the art will appreciate that values correlated to or proportional to, for example, the values listed in Table 9 are also useful to establish a reference gene expression profile. As used herein, “correlated” means that the values of the reference differential level of expression depart from independence of the values listed in Table 9 as evaluated by statistical methods known to those skilled in the art (see description further herein) to establish the relationship between the reference differential level of expression and the values listed in Table 9. As used herein, “proportional” means that the values of the reference differential level of expression follows a linear relationship with the values listed in Table 9, for example, by applying a linear model, such as, linear regression following common knowledge in the art.

Gene expression profiles may be “compared” by any of a variety of statistical analytic procedures. In particular, classifying an individual as having good or poor prognosis according to the above method may be performed by one skilled in the art by calculating a coefficient for correlation or distance or similarity after analyzing and comparing the gene expression profiles of sets of genes in the individual with the reference gene expression profile, including without limitation, differential expression profiles of corresponding sets of genes between two reference breast cell lines, wherein a first reference breast cell line endogenously expresses ZEB2 and wherein a second reference breast cell line only differs from the first reference breast cell line in that the expression of ZEB2 is knocked-down. Numerous methods for calculating a coefficient for correlation are well known for the one skilled in the art. Illustratively, the one skilled in the art may calculate a coefficient for correlation according to the Pearson, Spearman, or Kendall methods. Alternatively, the one skilled in the art may calculate a distance according to the Euclidian, Can berra, Manhattan, Maximum or Minkowski methods. The one skilled in the art may also calculate a similarity by using the inverse of the distance calculated according to the methods mentioned above. Within the present context, “coefficient for correlation” or “distance” or “similarity” is also referred to as “ZEB2 activity index.” It is meant that a patient will be assigned a poor/good prognosis with increasing/decreasing coefficient for correlation or similarity and a poor/good prognosis with decreasing/increasing distance. Thus, in the case the ZEB2 activity index is calculated as a coefficient for correlation or similarity, it is meant that a patient will be assigned a poor/good prognosis with high/low ZEB2 activity index. Otherwise, in the case the ZEB2 activity index is calculated as a distance, it is meant that a patient will be assigned a poor/good prognosis with low/high ZEB2 activity index.

As it is shown in the examples further herein, the inventors have identified prognostic ZEB2-associated gene expression profiles endowed with a high statistical relevance, with P values always below 0.05. Statistical relevancy of the above markers primarily selected was fully corroborated by Cox survival analysis, as it is shown in the examples herein. In certain embodiments, the prediction of relapse and/or recurrence of metastasis is expressed as a statistical value, including a P value, as calculated from the expression values obtained from the sets of genes that have been tested.

In a specific embodiment of the above method, the individual is classified as having a poor prognosis if the value obtained in step (iii) exceeds a certain threshold value, and the individual is classified as having a good prognosis if the value obtained in step (iii) is below a threshold value. Typically, the threshold value is the value providing the highest Chi squared value of a Cox survival analysis ran on a training set of patients, as it is shown in the examples further herein.

The inventors have also observed and verified that methods using the above-described ZEB2-associated gene expression profiles as a prognostic marker can achieve a sensitivity of 80% or more and/or a specificity of 80% or more. Hence, in an embodiment of the prognosis methods as taught herein, the sensitivity and/or specificity of the methods is at least 50%, at least 60%, at least 70% or at least 80%, e.g., at least 81%, at least 82%, at least 83%, at least 84%, at least 85%, at least 86%, at least 87%, at least 88%, at least 89%, at least 90%, or at least 95%. For example, between 80% and 100%, or between 81% and 95%, or between 83% and 90%, or between 84% and 89%, or between 85% and 88%.

Further, also described is a method for monitoring a change in the prognosis of an individual suffering from or suspected of suffering from breast cancer, the method comprising the steps of:

(i) applying any of the above methods to the individual at one or more successive time points, whereby the prognosis of breast cancer in the individual is determined at the successive time points;

(ii) comparing the prognosis of breast cancer in the individual at the successive time points as determined in (i); and

(iii) finding the presence or absence of a change between the prognosis of breast cancer in the individual at the successive time points as determined in (i).

In particular, the change in prognosis of breast cancer in the individual is monitored in the course of a medical treatment of the subject.

Monitoring the influence of agents (e.g., drug compounds) on the gene expression profile of the disclosure can be applied for monitoring the metastatic potency of the treated breast cancer of the patient with time. For example, the effectiveness of an agent to affect biological marker expression can be monitored during treatments of subjects receiving anti-cancer, and especially anti-metastasis, treatments.

In a preferred embodiment, provided is a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from an individual prior to administration of the agent; (ii) detecting the expression level of the sets of genes hereof in the pre-administration sample; (iii) obtaining one or more post-administration samples from the subject; (iv) detecting the expression level of the corresponding sets of genes in the post-administration samples; (v) comparing the expression levels of the sets of genes in the pre-administration sample with the expression level of sets of genes in the post-administration sample or samples; and (vi) altering the administration of the agent to the subject accordingly. Changes in gene expression profiles during the course of treatment may give information on effectiveness of dosage and the desirability of increasing/decreasing the dosage or may indicate efficacious treatment and no need to change dosage.

Performing the metastasis prediction method hereof may indicate, with more precision than the prior art methods, those patients at high-risk of tumor recurrence who may benefit from adjuvant therapy, including immunotherapy. For example, if, at the end of the metastasis prediction method hereof, a good prognosis of no metastasis is determined, then the subsequent anti-cancer treatment will not comprise any adjuvant chemotherapy. However, if, at the end of the metastasis prediction method hereof, a poor prognosis is determined, then the patient is administered with the appropriate composition of adjuvant chemotherapy.

The expression levels of the marker genes in a sample may be determined by any means known in the art. For example, the expression level may be determined by isolating and determining the level or the amount of nucleic acid transcribed from each marker gene. Alternatively, or additionally, the level of specific proteins translated from mRNA transcribed from a marker gene may be determined.

The level of expression of specific marker genes can be accomplished by determining the amount of mRNA, or polynucleotides derived therefrom, present in a sample according to conventional methods well known in the art. See, for example, Sambrook et al. 1989 and Ausubel et al. 1992. These examples are not intended to be limiting.

The terms “quantity,” “amount” and “level” are synonyms and generally well understood in the art. The term “as used herein” may particularly refer to an absolute quantification or a molecule or an analyte in a sample, or to a relative quantification of a molecule or analyte in a sample, i.e., relative to another value, such as, relative to a reference value as taught herein, or to a range of values indicating a base-line expression of a marker. These values or ranges can be obtained from a single patient or from a group of patients.

In certain embodiments, polynucleotide microarrays are used to measure expression so that the expression status of each of the markers above is assessed simultaneously. In a specific embodiment, provided are oligonucleotide or cDNA arrays comprising probes hybridizable to the genes corresponding to each of the marker gene sets of the gene signatures described above (i.e., markers to distinguish individuals with good prognosis versus individuals with poor prognosis). In a more specific embodiment, provided are oligonucleotide arrays comprising probes hybridizable to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 of the genes from Table 1.

As used herein, the term “probe” refers to any molecule, which is capable of selectively binding to a specifically intended target molecule, for example, a nucleotide transcript or protein encoded by or corresponding to a genetic marker. Probes can be synthesized by one skilled in the art. For example, the probe sequences can be synthesized enzymatically in vivo, enzymatically in vitro (e.g., by PCR), or non-enzymatically in vitro. For purposes of detection of the target molecule, probes may be specifically designed to be labeled, as described herein. Examples of molecules that can be used as probes include, but are not limited to, RNA, DNA, protein, antibodies, and organic molecules. In some embodiments, probes are polynucleotides complementary to or homologous with at least a portion (e.g., at least 7, 10, 15, 25, 30, 40, 50, 100, 500, or more nucleotide residues) of a biological marker nucleic acid or gene. The terms “polynucleotide,” “oligonucleotide,” “polynucleic acid,” “nucleic acid” are interchangeably used herein and are known to the one skilled in the art.

In specific embodiments, provided are polynucleotide arrays in which polynucleotide probes complementary and hybridizable to the breast cancer prognosis-related markers, described herein, are at least 50%, 60%, 70%, 80%, 85%, 90%, 95% or 98% of the probes on the array. In another specific embodiment, the microarray comprises probes to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 genes selected from Table 1. Preferably, the microarray comprises probes to all 35 genes listed in Table 1. In some embodiments, a microarray hereof comprises probes to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 genes from Table 5. Preferably, the microarray comprises probes to at least 2, 3, 4, 5, 6, 7, 8, 9, 10, 11, 12, 13, 14, 15 or 16 genes from Table 7. In more preferred embodiments, the microarray comprises probes to each of the 16 genes listed in Table 7. According to a particular preferred embodiment, the microarrays, as described hereinabove, are further characterized in that they at least comprise one or more probes to ZEB2.

An exciting prospect of microarray-based tests is that multiple, distinct predictions, including prognosis, ER and HER2 status, and sensitivity to various treatment approaches, can be generated from a single assay. This type of test may use information from different sets of genes from the same tissue for different predictions. Accordingly, the microarray may additionally include sets of probes complementary and hybridizable to genes informative for related or unrelated conditions. For example, a microarray may additionally comprise probes complementary and hybridizable to genes informative for ER tumor status, genes that may be used to distinguish sporadic from BRCA-I type tumors, or genes that are informative for any other clinical aspect of breast cancer, or any other related or unrelated condition.

General methods pertaining to the construction of microarrays comprising the probes and/or subsets above are described in the following sections.

Microarrays are prepared by selecting probes, which comprise a polynucleotide sequence, and then immobilizing such probes to a solid support or surface, which may be either porous or non-porous. For example, the probes may be polynucleotide sequences, which are attached to a nitrocellulose or nylon membrane or filter covalently at either the 3′ or the 5′ end of the polynucleotide. Such hybridization probes are well known in the art (see, e.g., Sambrook et al. 1989). Alternatively, the solid support or surface may be a glass or plastic surface.

In preferred embodiments, a microarray comprises a support or surface with an ordered array of binding (e.g., hybridization) sites or probes each representing one of the genetic markers described herein. Specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site. The microarrays of the disclosure include one or more test probes, each of which has a polynucleotide sequence that is complementary to a subsequence of RNA or DNA to be detected. Preferably, the position of each probe on the solid surface is known.

Microarrays can be made in a number of ways, and non-limiting examples are described further below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm and 25 cm, between 12 cm and 13 cm, or 3 cm. However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of genomic DNA or cloned sequences, and is well known in the art. An alternative, preferred means for generating the polynucleotide probes of the microarray is by synthesis of synthetic polynucleotides or oligonucleotides. In some embodiments, synthetic nucleic acids include non-natural bases, such as, but by no means limited to, inosine.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array.

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al. (1995a). This method is especially useful for preparing microarrays of cDNA (see also, DeRisi et al. 1996; Shalon et al. 1996; and Schena et al. 1995b).

Another preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al. 1991; Pease et al. 1994; Lockhart et al. 1996; U.S. Pat. Nos. 5,578,832; 5,556,752; and 5,510,270) or other methods for rapid synthesis and deposition of defined oligonucleotides. When these methods are used, oligonucleotides (e.g., 60-mers) of known sequence are synthesized directly on a surface, such as, a derivatized glass slide. Usually, the array produced is redundant, with several oligonucleotide molecules per RNA.

The polynucleotide molecules, which may be analyzed as per the disclosure (the “target polynucleotide molecules”) may be from any clinically relevant source, but are expressed RNA or a nucleic acid derived therefrom (e.g., cDNA). In one embodiment, the target polynucleotide molecules comprise RNA, including, but by no means limited to, total cellular RNA, poly(A)+ messenger RNA (mRNA) or fraction thereof, cytoplasmic mRNA, or RNA transcribed from cDNA. Methods for preparing RNA are well known in the art, and are described generally, e.g., in Sambrook et al. 1989. In one embodiment, RNA can be fragmented by methods known in the art, e.g., by incubation with ZnCl2, to generate fragments of RNA. In another embodiment, the polynucleotide molecules analyzed hereby comprise cDNA, or PCR products of amplified RNA or cDNA.

As described above, the target polynucleotides are detectably labeled at one or more nucleotides according to any method known in the art. Preferably, this labeling incorporates the label uniformly along the length of the RNA. In a preferred embodiment, the detectable label is a luminescent label. For example, fluorescent labels, bioluminescent labels, chemiluminescent labels, and colorimetric labels may be used in the disclosure. In a highly preferred embodiment, the label is a fluorescent label, such as, a fluorescein, a phosphor, a rhodamine, or a polymethine dye derivative. Examples of commercially available fluorescent labels include, for example, fluorescent phosphoramidites, such as, FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a reference or standard. In the context of the disclosure, the reference may comprise target polynucleotide molecules from two reference breast cell lines, wherein a first reference breast cell line endogeneously expresses ZEB2 and wherein a second reference breast cell line only differs from the first reference in that the expression of ZEB2 is knocked-down. In this embodiment, target polynucleotide molecules from the two reference breast cell lines are differentially labeled. In another embodiment, the target polynucleotide molecules are derived from the same individual, but are taken at different time points, and thus indicate the efficacy of a treatment by a change in expression of the markers, or lack thereof, during and after the course of treatment (i.e., chemotherapy, radiation therapy or cryotherapy), wherein a change in the expression of the markers from a poor prognosis pattern to a good prognosis pattern indicates that the treatment is efficacious. In this embodiment, different time points are differentially labeled.

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al. (1989), and in Ausubel et al. (1992). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al. 1993).

When fluorescently labeled probes are used, the fluorescence emissions at each site of a microarray may be, preferably, detected by scanning confocal laser microscopy. In one embodiment, a separate scan, using the appropriate excitation line, is carried out for each of the different fluorophores used. Alternatively, a laser may be used that allows simultaneous specimen illumination at wavelengths specific to the different fluorophores and emissions from the different fluorophores can be analyzed simultaneously. In a preferred embodiment, the arrays are scanned with a laser fluorescent scanner. Fluorescence laser scanning devices are described in Schena et al. (1996), and in other references cited herein. Alternatively, the fiber-optic bundle described by Ferguson et al. (1996), may be used to monitor mRNA abundance levels at a large number of sites simultaneously. Signals are recorded and, in a preferred embodiment, analyzed by computer.

Quantitative reverse transcriptase PCR (quantitative RT-PCR or qRT-PCR) can also be used to determine the expression level of a marker gene. The first step in gene expression profiling by RT-PCR is the reverse transcription of the RNA template into cDNA, followed by its exponential amplification in a PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using, e.g., commercially available equipment, such as, for example, ABI PRISM 7700™. SEQUENCE DETECTION SYSEM™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device, such as, the ABI PRISM 7700™ SEQUENCE DETECTION SYSTEMT™.

As an alternative, Sybr Green technology can also be used, as is described in the example section.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and 13-actin.

A more recent variation of the RT-PCR technique is the real time quantitative PCR, which measures PCR product accumulation through a dual-labeled fluorigenic probe (i.e., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g., Held et al. 1996.

The gene expression profile and/or the expression levels of the marker genes according to the disclosure may be expressed as any arbitrary unit that reflects the amount of the corresponding mRNA of interest that has been detected in the tissue sample, such as, intensity of a radioactive or of a fluorescence signal emitted by the cDNA material generated by PCR analysis of the mRNA content of the tissue sample, including (i) by Real-time PCR analysis of the mRNA content of the tissue sample and (ii) hybridization of the amplified nucleic acids to DNA microarrays.

In a particular embodiment, it is possible to determine a corresponding protein expression profile based on the identified gene expression profile. A protein expression profile can conveniently be detected by the use of specific antibodies directed against the differentially expressed protein products. Illustratively, the proteins from a sample can be separated on a polyacrylamide gel, followed by identification of specific marker-derived proteins using antibodies in a western blot. Alternatively, proteins can be separated by two-dimensional gel electrophoresis systems. Two-dimensional gel electrophoresis is well known in the art and typically involves isoelectric focusing along a first dimension followed by SDS-PAGE electrophoresis along a second dimension. The resulting electropherograms can be analyzed by numerous techniques, including mass spectrometric techniques, western blotting and immunoblot analysis using polyclonal and monoclonal antibodies. See, for example, Harlow and Lane (1990).

In another aspect, provided is a kit useful for detecting the gene expression profile hereof In one embodiment, a kit is provided for measuring the expression levels of a plurality of genes comprising the necessary tools and equipment. For example, a kit to carry out a PCR analysis, preferably a multiplex PCR analysis, such as, a multiplex RT-PCR analysis, comprises a combination of reagents, such as, primers, buffers, polynucleotides and a thermostable DNA polymerase. In a preferred embodiment, the kit contains a microarray ready for hybridization to target polynucleotide molecules. The kits, as herein described, may also comprise reference sample material. In addition, provided is a kit for monitoring the effectiveness of treatment of an individual with an agent, which kit comprises means for quantifying the expression levels of the sets of genes hereof that is indicative of the probability of occurrence of metastasis in the individual suffering from breast cancer. The kits hereof, can be used in clinical settings or at home.

In still another aspect of the disclosure, a gene expression profile indicative for a good prognosis or a poor prognosis of an individual suffering from or suspected of suffering from breast cancer is also provided, the gene expression profile comprising a quantified expression level of a plurality of genes comprising any combination of at least 8 genes from Table 1. In a particular embodiment, the gene expression profile is established by quantifying the expression level of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 member genes from Table 1. More specifically, the plurality of genes can be selected from the group comprising ANK2, ANK3, CADPS2, CASP1, CCND2, COL6A3, CXorf57, EDNRA, EFNB2, ENOX2, GAD1, HES1, IGFBP1, IL7, JAG1, KRT15, LTBP1, MAP3K5, MFAP3L, NDP, OASL, PDE2A, PLA2G4A, PORCN, RGS4, SCG5, SLC22A3, STC1, TBC1D8B, TCN1, THBD, TPK1, VNN1, XK and ZEB2. Preferably, the gene expression profile is established by quantifying the expression level of a plurality of genes comprising any combination of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 genes from Table 5. More specifically, the plurality of genes can be selected from the group comprising ANK2, ANK3, CADPS2, CCND2, COL6A3, CXorf57, EDNRA, EFNB2, ENOX2, GAD1, HES1, IGFBP1, IL7, JAG1, KRT15, LTBP1, MAP3K5, MFAP3L, NDP, OASL, PDE2A, PLA2G4A, PORCN, RGS4, SCG5, STC1, TBC1D8B, TCN1, THBD, TPK1, VNN1, XK and ZEB2. In more preferred embodiments, a gene expression profile can be determined by quantifying the expression level of a plurality of genes comprising any combination of at least 2, 3, 4, 5, 6, 7, 8, 9, 10, 11, 12, 13, 14, 15 or 16 genes from Table 7. More specifically, the plurality of genes can be selected from the group comprising ANK2, ANK3, CADPS2, CCND2, COL6A3, CXorf57, HES1, NDP, OASL, PLA2G4A, PORCN, RGS4, SCG5, TPK1, XK and ZEB2. In more preferred embodiments, a gene expression profile can be determined by quantifying the expression level of a plurality of genes comprising each of the following genes: ANK2, ANK3, CADPS2, CCND2, COL6A3, CXorf57, HES1, NDP, OASL, PLA2G4A, PORCN, RGS4, SCG5, TPK1, XK and ZEB2. It is understood that a gene expression profile can be further refined and optimized as presented in the example section. According to a particular preferred embodiment, the gene expression profile is determined by quantifying the expression level of a plurality of genes as described above, further characterized in that at least ZEB2 is comprised within the plurality of genes. Or in other words, that ZEB2 is a member gene of the gene expression profile as defined hereinbefore.

Further, a reference gene expression profile, as defined hereinbefore, is also encompassed in the disclosure.

In another embodiment, the hereinbefore defined gene expression profiles, may be used for the prognosis of an individual suffering from or suspected of suffering from breast cancer according to the methods described herein. It is to be understood that, by using the same methodology as described above and/or in the example section, additional gene expression profiles can be generated based on the transcriptional activity of other genes, for example, other EMT inducers, such as, ZEB1. Thus, in order to further increase the predictive value of gene expression profiles for relapse risk in breast cancer patients, a combination of two or more gene expression signatures can be used.

The following examples are intended to promote a further understanding of the disclosure. While the disclosure is described herein with reference to illustrated embodiments, it should be understood that the disclosure is not limited hereto. Those having ordinary skill in the art and access to the teachings herein will recognize additional modifications and embodiments within the scope thereof.

### EXAMPLES

Materials and Methods to the Examples

Cell Lines and Cell Culture

Human MDA-MB-231 breast carcinoma cell line was obtained from the American Type Tissue Collection. Cells were maintained in Leibovitz-15 with 10% FCS, 200 nM L-glutamine and 100 μg/ml penicillin and 100 μg/ml streptomycin.

Transfection of Small Interfering RNAs

Two 19-nt-specific sequences were selected in the coding sequence of SIP1 to generate 21-nt sense and 21-nt antisense strands of the type (19N) TT (N, any nucleotide). The sense and antisense strands were then annealed to obtain duplexes with identical 3′ overhangs. The sequences were submitted to a BLAST search against the human genome to ensure the specificity of the small interfering RNA (siRNA) to the targeted sequence. Two duplexes that do not recognize any sequence in the human genome were used as controls. The 19-nt-specific sequences for the two ZEB2/SIP1 siRNAs are as follows: ZEB2/SIP1 Sil, 5′-GUAAUCGCAAGUUCAAAU-3′ (SEQ ID NO:1); ZEB2/SIP1 Sit, 5′-GAACAGACAGGCUUACUUA-3′ (SEQ ID NO:2). For transfection of the siRNA duplexes, 75 000 cells were plated in six-well plates containing 2 ml of culture medium per well. After 24 h the cells were transfected by the calcium phosphate precipitation method: into each well were added 200 ml of a mixture containing 20 nMsiRNA duplexes, 140 mMNaCl, 0.75 mM Na2HPO4, 6 mM glucose, 5 mMKCl, 25 mM HEPES and 125 mM CaCl2. Twenty-four hours later, the cells were extensively washed with PBS, incubated for 48 h in culture medium, and then harvested for RT-PCR or Western blotting analysis. An FITC-labeled control siRNA (Eurogentec, Belgium) was also transformed in parallel and revealed an uptake of the siRNA in 100% of the cells

Construction and Transduction of Short Hairpin-Containing Lentiviral Vectors

A ZEB2/SIP1-specific siRNA sequence was designed using selection criteria as described (Brummelkamp et al. 2002; Ui-Tei et al. 2004). A double PCR approach was used to create anshRNA expression cassette, which was cloned in the lentiviralpLVTH vector (Wiznerowicz and Trono 2003) using EcoRI and ClaI restriction sites. The primers for the first PCR were 5′-CTGCAGGAATTCGAACGCTGACGTCATCAA-3′ (SEQ ID NO:3) and 5′-AAATCTCTTGAATTTAACAATACCCAGCTCCGGGGATCTGTGGTCTCATACAGAA CTTATAA-3′ (SEQ ID NO:4). This PCR product was a template for a second PCR reaction with the same forward primer and the reverse primer 5′-CCATCGATAAGCTTTTTTTCCAAAAAAGGAGCTGGGTATTGTTAAATCTCTTGAAT TTA-3′ (SEQ ID NO:5).

For lentivirus production, 1.2 million cells of the packaging cell line HEK293T were seeded in a 25-cm2 flask. After 24 h, 3 mg of the pLV-THshRNA construct or empty vector, 3 mg of the packaging plasmid CMVdR8.91 and 1.5 mg of the envelope plasmid pMD2G-VSVG were first precipitated together and then transfected into the HEK293T cells using the calcium phosphate precipitation method. The DNA was premixed with 50 ml of 2 M CaCl2 and 190 ml TE buffer and then slowly added to 250 ml HBS. The mixture was put on a shaker for 15 min before it was added to the cells. After 8 h, the cells were washed and incubated for 48 h in 4 ml fresh culture medium. The virus-containing medium was then harvested and filtered through a 0.45-mm low-protein-binding filter (Millipore, Billerica, Mass., USA). Aliquots were stored at −70° C. Transduction of the MDA-MB-231 cells was performed by mixing 50 000 cells with 200 μl viral supernatant in a 96-well plate, and three replicates of each transduction were made. These mixtures were centrifuged for 1.5 h at 32° C. and 1500 rpm before incubating them at 37° C. After 24 h, the cells were trypsinized and replicates were pooled in a 24-well plate together with 800 μl fresh viral supernatant. The mixtures were again centrifuged as mentioned above and incubated for 24 h, and then the medium was replaced with fresh culture medium. Transduction efficiencies were determined by measuring EGFP expression using FACS analysis (Epics Altra, Beckman Coulter, Fullerton, Calif., USA). Subsequently, the cells were sorted to obtain cell populations with more than 90% EGFP-positive cells.

Real Time Quantitative RT-PCR

Primers and probes for qRT-PCR were designed using primer Express qRT-PCR 1.0 Software (Perkin Elmer Applied Biosystems). cDNA synthesis and PCR amplification were described previously as were the primer and probe sequences for human ZEB2/SIP1, E-cadherin and N-cadherin (Vandewalle et al. 2005). Sequences of primers for ZEB1/δEF1 were 5′-TGTTACCAGGGAGGAGCAGTG-3′ (SEQ ID NO:6) and 5′-TCTTGCCCTTCCTTTCTGTCA-3′ (SEQ ID NO:7). The primers and probe for Snail were 5′-CAGGACTCTAATCCAGAGTTTACCTTC-3′ (SEQ ID NO:8), 5′-GGGATGGCTGCCAGCA-3′ (SEQ ID NO:9) and 5′-FAM-AGCAGCCCTACGACCAGGCCCA-TAMRA-3′ (SEQ ID NO:10). The primers and probe for Slug were 5′-GCCAAACTACAGCGAACTGGA-3′ (SEQ ID NO:11), 5′-TGTGGTATGACAGGCATGGAG-3′ (SEQ ID NO:12) and 5′-FAM-CACATACAGTGATTATTTCCCCGTATCTCTA-TAMRA-3′ (SEQ ID. NO:13). (FAM is 6-FAMT™ and TAMRA is TAMRA™, both are dyes).

AffymetrixGeneChip Analysis

The microarray experiment was performed as described before (Vandewalle et al. 2005; Perou et al. 2000) at the VIB MicroArray facility (MAF), including probe labeling and hybridization on AffymetrixGeneChip (Human Genome U133 Plus 2.0) and subsequent data acquisition and processing. A gene was scored as down-regulated if AvRatio<0.5 and up-regulated if AvRatio>2 in the case of stable knock-down and as down-regulated if AvRatio<0.75 and up-regulated if AvRatio>1.25 in the case of transient knock-down. The microarray data obtained within this study can be viewed on the NCBI-GEO website (worldwide web at ncbi.nlm.nih.gov/geo) with the accession number GSE27966.

ZEB2 Expression Analysis in Human Primary Breast Cancers

cDNA was synthesized from 2.5-μg samples of total RNA using the IscriptcDNA synthesis kit (Bio-Rad). Subsequently qPCR on the LC480 (Roche) was done for ZEB2 and different reference genes using LC 480 Sybr Green I master kit (Roche), Fast SYBR master mix kit (Applied Biosystems), and Taqman fast universal. PCR Mastermix (Applied Biosystems). By using GeNorm (Vandesompele et al. 2002), we determined the most accurate set of reference genes for normalization (HMBS, SDHA, TBP and UBC). The average threshold cycle of triplicate reactions was used for all subsequent calculations using the delta Ct method. Relative ZEB2 expression levels (average of 10 samples with low expression set to 1) were depicted in descending order.

Microarray Data Analysis

Probe sets of good reliability were next selected based on consistency of annotation in the Geneannot (worldwide web at bioinfo2.weizmann.acil/cgi-bin/home_page.pl) or PLANdbAffy (worldwide web at affymetrix2.bioinf.fbb.msu.ru/) databases and on the reproducibility of the expression values corresponding to common breast cancer cell lines described in the studies GSE10890, GSE12777 and GSE16795. A probe set was considered as reliable when both the corresponding Geneannot annotation quality, the specificity and the sensitivity indexes were all equal to one. A probe set was considered as reliable when more than 63% of the probes from the probe sets are flagged as green (perfect match) or yellow (perfect match but with sequence in non-coding RNA) in the PLANdbAffy database. To evaluate reproducibility, the expression values for each probe set observed in the common cell lines in one study were linearly correlated to the corresponding values described in the two other studies. A probe set was considered as reliable if the averaged Pearson correlation coefficient is above 0.5. To compare, within one study, the cell line expression values corresponding to different probes, the intensity values for each probe set were normalized by removing the minimal intensity value considered as background and dividing these values by the range of intensities.

We downloaded the cell files of nine studies performed on affymetrix array platforms compatible with our ZEB2 knock-down data (HG133A or HG133plus2) involving at least 20 breast cancer cell lines (Table 1) and tumor samples (Table 2) published before September 2009 in the GEO or Array Express databases. Data were extracted, background-subtracted, normalized and summarized (median polish option) using frozen RMA, the new summarization bioconductor package developed by Dr. Irizarry's group (McCall et al. 2010). This package estimates and corrects probe-specific effects and variance on the basis of a common vector defined using all data obtained with the same platform as that used to generate the analyzed data published in GEO. Data expressed by default in a log scale by the IRMA script were converted back to signal intensity values. Data extraction, processing, analysis and display were performed using R scripts. To enhance the statistical power of our analysis, we merged the data from the nine studies into a single pooled database. This was possible because the same expression values for each probe set are obtained for the same patient when IRMA summarized expression data are considered. By comparing the patient names, we realized that several patients were included in two or more studies. The identity of these patients was confirmed if the patients' identification numbers were identical and the clinical parameters and expression data were the same in the different studies. To avoid over-fitting, those patients were included only once in the pooled database. Patients with no data on occurrence or time of relapse were removed from the pooled database. Furthermore, we noticed that the distribution of the patients in the different studies in the categories of relapse/no relapse or among the molecular subtypes is not balanced. For example, by design, only about 10% of the patients in the GSE12276 study had not relapsed, because the goal of the study was to evaluate the relationship between gene expression profile and site of relapse. Hence, different subsets of the database were generated according to the platform used and the relative contributions of the breast cancer subtypes within the individual studies (Table 10). The relationship between the patient names, GEO identification numbers and inclusion in the different studies or selection lists is documented in an excel file (data not shown). The criteria used to include patients from the different studies in the different selections are described in Table 11.

Heatmaps were drawn with the heatmap.2 function of the R package gplots, using the normalized intensity values, the Spearman correlation coefficient as distance metric, and the average clustering method. Cox survival analyses were performed in R with the Survival package using raw expression intensity values or intensity data stratified in quarters or in dichotomic categories. For the stratification in quarters, the range of expression values was divided in four equal intervals before each expression intensity value was assigned a value of 1, 2, 3 or 4 according to the interval in which it fell. Dichotomic categories are defined as 0 or 1, depending on whether or not expression the value is above a threshold value leading to the highest Chi-square value in the training Cox survival analysis.

We selected 36 probe sets of good quality commonly down-regulated upon transient or stable ZEB2 knock-down in MDAMB231 cells (Table 5). For each probe set, differences between the expression value in the MDAMB231 cells with or without knock-down of ZEB2 were correlated, for each patient in the nine selected studies, with the expression values of the corresponding selected probe sets. The original probe set list was next optimized by two steps. First, we iteratively removed all the probe sets one by one, except the ZEB2 probe set from the initial list. Second, at every iteration step, we selected the list with the highest Chi-squared value for data expressed as quarters or dichotomic categories, or, the highest normalized relative risk ratio in the case of data expressed as raw values. Different optimized lists were obtained when the raw ZEB2 activity index or the ZEB2 activity index was stratified in quarters or in dichotomic categories, respectively, and the pooled dataset was used as input in the Cox analysis. Because we noticed that the distribution of patients from the different studies in the relapse/no relapse categories or the molecular subtypes is not balanced, we performed the list optimization procedure with the six different patient sets described in Table 10 according to the patient inclusion criteria defined in Table 11. We thereby generated a number of lists of 10 to 24 probe sets according to the patient set used or the way that the ZEB2 activity index is expressed, as described in Table 12. For each of those lists, consistency of the association of the ZEB2 activity index with risk of relapse was checked by cross-validation using the raw ZEB2 activity index of the ZEB2 activity index stratified in quarters or in dichotomic categories as input for each of the selected patient sets and for the reference patient set (named Patsel). Patients from the pooled data series were randomly distributed into a training set comprising 75% (n=1050 in the reference set) of the samples and a complementary validation set comprising of the remaining samples (n=350 in the reference set). The selection procedure was performed in parallel with both the training and validation sets. Stability of the probe set selection and reproducibility of the Cox p-values and relative risk coefficients were analyzed upon 100 iterations. The ZEB2 activity index is considered stable if it is significantly associated (at the 0.05 level) with increased risk in 100% of the training sets and more than 85% of the validation sets. Finally, we compared the hazard ratio, the p-value, and the stability in cross-validation analysis of ZEB2 activity indexes corresponding to each list when their raw values or their quarters-stratified or dual-categories-stratified values obtained with the different patient sets were used as input. Stability of the ZEB2 activity index was further evaluated by comparing its performance between each study analyzed individually (Table 5). For each patient set, the accuracy of the prediction based on the ZEB2 activity index stratified in dichotomic categories was estimated by evaluating its sensitivity and specificity. Sensitivity is defined as the proportion of relapsing patients predicted to relapse. Specificity is defined as the proportion of patients who did not relapse and who were assigned a low probability of relapse. As illustrated in Table 13, we selected for further analysis the shortest list (List3P6; ZEB2AI16) that fulfilled six criteria irrespectively of the way the ZEB2 activity index was expressed. First, that it led the most often to a ZEB2 activity index that was significantly associated with increased relapse risk when each study was evaluated individually (counts of studies with increased hazard and logrank test p-value below 0.05). Second, that it provided the most stable results in cross-validation analysis run on the different pooled patient datasets (counts of patient sets with increased hazard in 100% of the training sets and more than 85% of the validation sets). Third, that it led the most often to a ZEB2 activity index that was significantly associated with increased relapse risk in the different patient sets (counts of sets with increased hazard and logrank test p-value below 0.05). Fourth, that it led the most often in the different patient sets to a ZEB2 activity index sensitivity above 0.3 when the specificity was above 0.85 (counts of sets). Fifth, that it led to an average sensitivity above 0.3 and an average specificity above 0.85. Sixth, that it led the most often to a Fisher's exact test p-value below 0.05 (counts of patient sets).

To compare the gene expression profiles of ZEB2-depleted MDA-MB-231 cells with the profiles of different malignant mammary cell populations, including putative breast cancer stem cell populations, we extracted and processed the corresponding micro-array data published in GEO. We extracted the expression values corresponding to the probe sets of the selected genes affected by ZEB2 depletion as well as of the markers used to isolate the different populations of breast cancer cells.

### Example 1

### ZEB2/SIP1 Transcription Factor is Strongly Expressed in Basal Breast Cancer Cell Lines

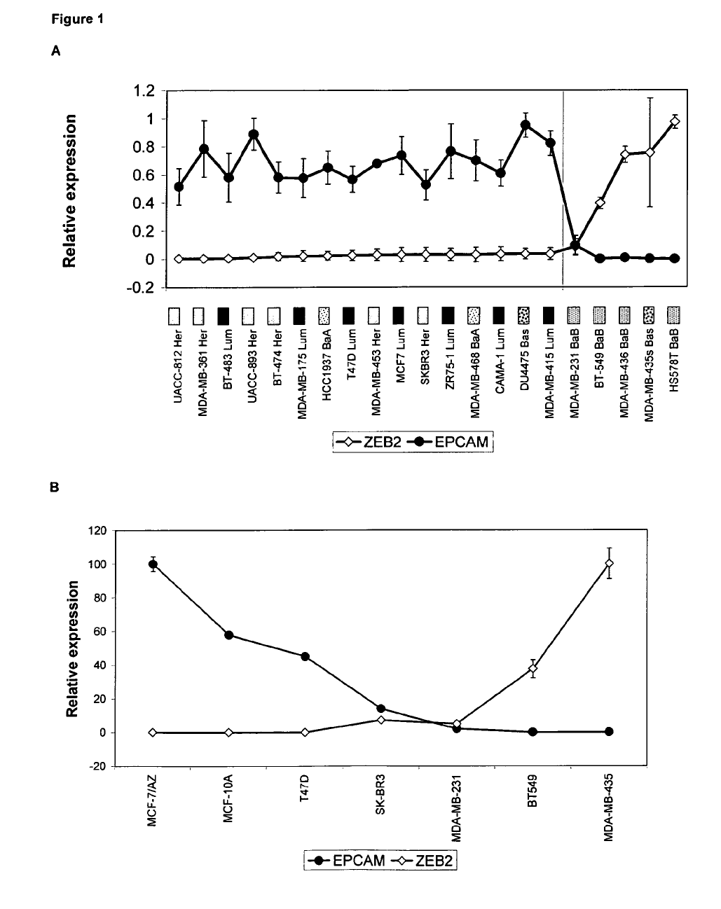

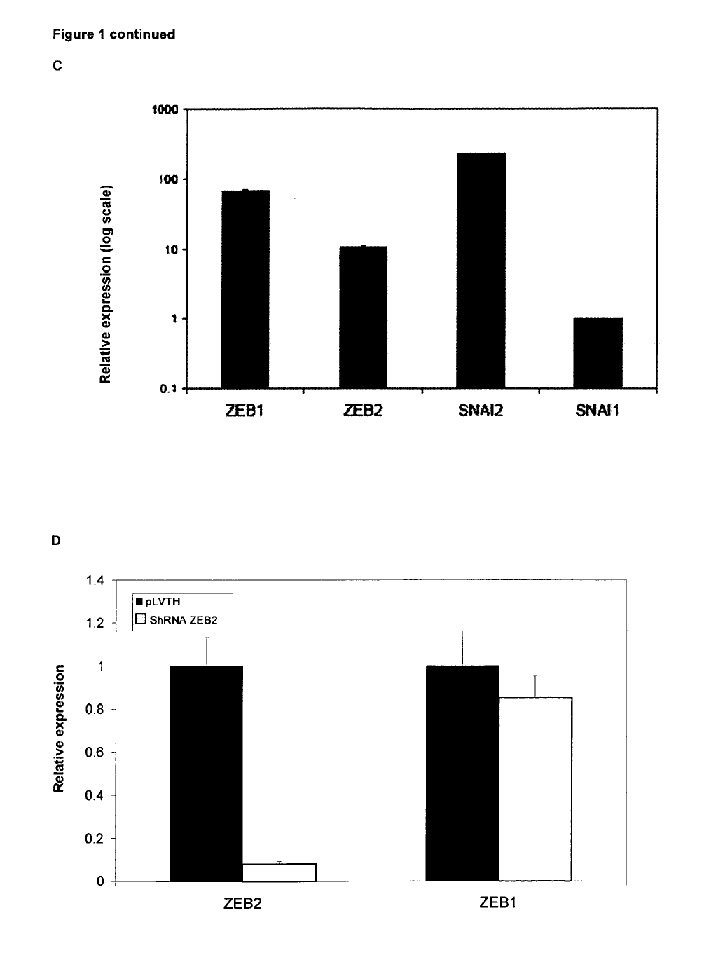

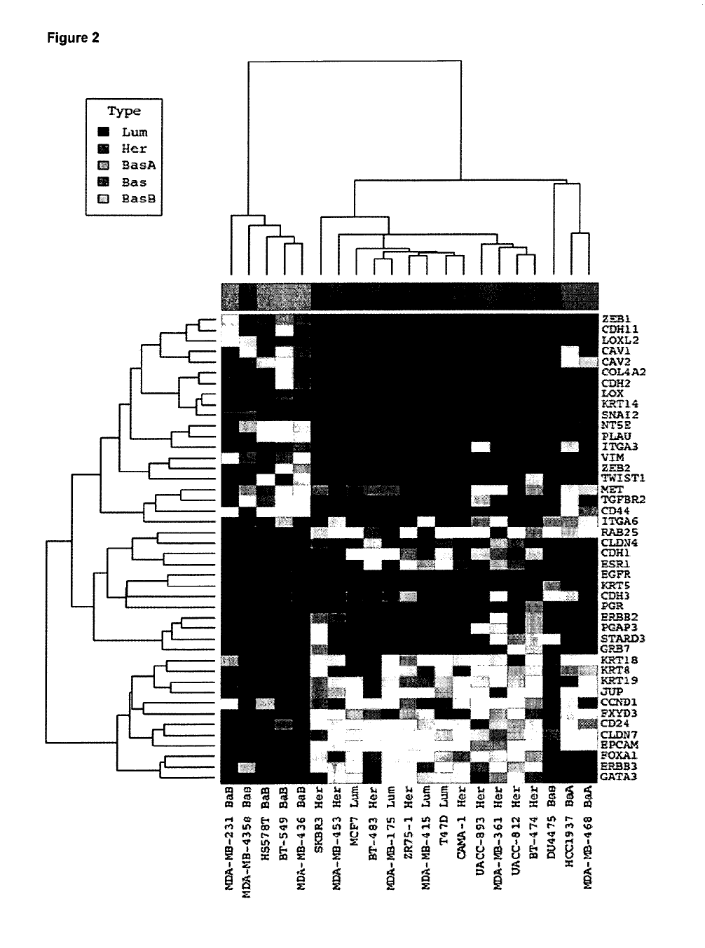

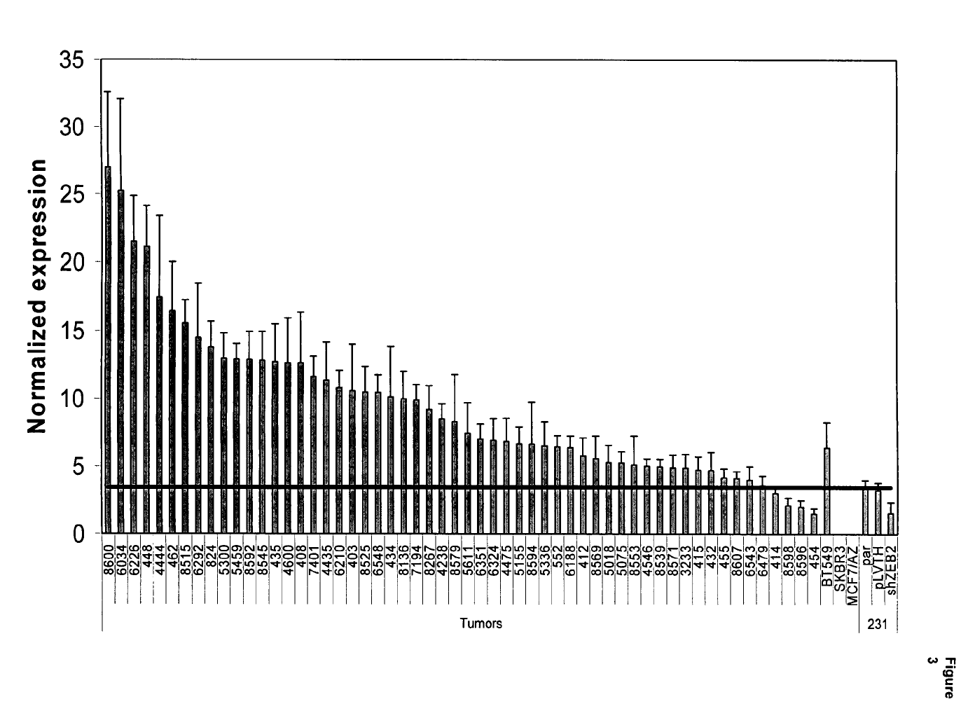

Breast cancer is a heterogeneous disease with at least five “intrinsic” subtypes defined on the basis of gene expression profiles (Perou et al. 2000; Sorlie et al. 2001; Sotiriou et al. 2006). Interestingly, breast cancer cell lines can also be segregated in similar classes according to their gene expression profiles (Neve et al. 2006). To identify cellular models with elevated ZEB2/SIP1 expression and define their gene expression profiles, we downloaded the gene expression data of studies involving at least 20 breast cancer cell lines (Table 2). We compared the ZEB2 expression intensity levels of all cell lines in each study with the corresponding EPCAM expression values, used as marker of epithelial differentiation. After ranking the cell lines according to ZEB2 level, we observed that ZEB2 starts to increase in MDAMB231 breast cancer cells and reaches a maximum in Hs578T cells (FIG. 1A). Interestingly, expression of EPCAM starts to drop as soon as ZEB2 starts to increase. This relationship between ZEB2 expression and epithelial markers was confirmed by quantitative RT-PCR (FIG. 1B). Moreover, ZEB2 seems to be expressed mainly in the basal-like type of cell as defined by Neve and collaborators (Neve et al. 2006). In addition, MDAMB231 breast cancer cells share many features of mesenchymal cells, including loss of E-cadherin expression and gain of vimentin with other basal cells (FIG. 2). Furthermore, we determined by quantitative RT-PCR that among the other E-cadherin repressors, SNAI2 showed the highest expression level in MDA-MB-231, followed by ZEB1/δEF1, while ZEB2 expression was intermediate and SNAI1 expression was moderate (FIG. 1C). TWIST1 expression was undetectable.

### Example 2

### Gene Expression Patterns are Altered in MDAMB231 Cells Upon ZEB2/SIP 1 Knock-Down

To create a stable ZEB2/SIP1 knock-down derivative of the MDAMB231 cell line, these cells were infected with a lentiviral vector (Wiznerowicz and Trono 2003) containing an anti ZEB2 short hairpin RNA sequence-ires-GFP and sorted for GFP-positive cell populations. Quantitative RT-PCR analysis of these cell populations showed that ZEB2/SIP1 mRNA expression in MDAMB231 derivatives infected with the ZEB2-targetting lentivirus was more than 90% lower than in control cells transduced with the empty vector (pLVTH). Importantly, thanks to the weak sequence similarity of the 3′UTR sequences of ZEB2/SIP1 and ZEB1/δEF1 to which the ZEB2/SIP1 shRNA is targeted, no reduction of the expression level of the closely related family member of ZEB2/SIP 1 could be detected (FIG. 1D), confirming the specificity of knock-down.

To document the changes in gene expression that coincide with loss of ZEB2/SIP1 activity in MDAMB231, we performed a transcriptome-wide differential gene expression survey using AffymetrixGeneChip arrays (see section: Material and Methods to the examples). cDNA of pooled MDAMB231 cells infected with the control pLVTH vector was compared to cDNA of pooled pLVTH-ZEB2-transduced MDAMB231. On the other hand, to avoid possible off-target effects and to shed light on primary ZEB2 targets, we also compared cDNA from mock transfected MDAMB231 cells to cDNA of MDAMB231 cells transfected with siRNA pools against ZEB2. Respectively, 8162 and 8314 probe sets fulfilled our quality control criteria in the stable and transient ZEB2 knock-down experiments. Of these probe sets, 283 were up-regulated and 204 were down-regulated at least twofold upon stable ZEB2 knock-down. On the other hand, only 3 and 14 probe sets were respectively up- or down-regulated at least twofold upon transient ZEB2 knock-down. Thirty-nine (39) probe sets were shared between the 204 and 503 probe sets down-regulated by at least 0.75-fold in the transient and at least 0.5-fold in the stable ZEB2 knock-down experiments, respectively, and corresponded to 35 genes with decreased expression upon ZEB2 knock-down (Table 1).

### Example 3

### ZEB2-Associated Alteration of Gene Expression Patterns (ZEB2 Metagene) Predicts Probability of Survival in Human Breast Cancer Clinical Studies

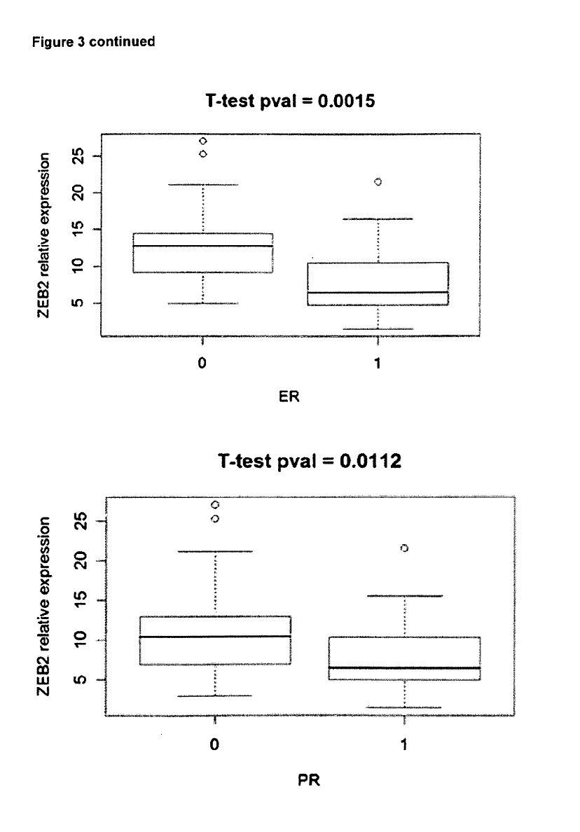

In the light of our in vitro and in vivo data, we wondered whether ZEB2 expression in breast tumors is associated with clinical parameters. To this end, we measured by quantitative RT-PCR the expression of ZEB2 in a pilot cohort of 56 breast tumor samples for which clinical parameters were available. As shown in FIG. 3, expression of ZEB2 in tumor samples is often higher than in breast cancer cell lines, significantly lower in ER/PR positive tumors. Next, we analyzed the gene expression data and associated clinical data of nine breast cancer clinical studies performed on the Affymetrix HG133 platforms compatible with our micro-array data for which relapse data are available (Table 3). Based on the gene expression changes induced upon ZEB2 knock-down in MDAMB231 and on probe set quality parameters, we selected 36 unique probe sets out of the 39 probe sets down-regulated upon both transient and stable ZEB2 depletion in the MDA-MB-231 cells (Table 5). These probe sets specifically measure the expression levels of 33 genes, corresponding to positive ZEB2 regulated genes (with reduced expression upon ZEB2 depletion). They fulfill our probe set quality control criteria as defined in Material and Methods to the examples. However, none of the expression values corresponding to these probe sets, including the probe set for ZEB2 (203603_s_at), is associated with a consistent, reproducible and significant change in relapse-free survival probability in the nine studies analyzed (Table 5).

In tumors, ZEB2 is expressed not only by malignant cells, but also to various degrees by accessory cells, such as, immune cells or endothelial cells also known to affect tumor progression (Lanigan et al. 2007). So, we wondered whether the relative changes in gene expression profiles associated with ZEB2 activity in the cancer cells would not be a better predictive marker than the absolute ZEB2 expression level of the tumor. In practice, we wanted to determine which tumors present a gene expression profile most similar to a corresponding reference gene expression profile linked to ZEB2 activity in a reference model of aggressive breast cancer cell line. In particular, we defined as a reference gene expression profile the difference between the expression values for the 36 selected probe sets corresponding to the 35 positive ZEB2 regulated genes of the wild-type cells and those of the pooled ZEB2 knocked-down MDAMB231 cells to the expression of the corresponding probe sets in each patient. For each patient sample, we defined the ZEB2 activity index as the Spearman coefficient for correlation between the selected probe sets expression values in the tumor samples and the corresponding ZEB2 knocked-down MDAMB231 reference. In other words, this index measures the distance between the expression profiles of ZEB2 regulated genes of an archetype of basal-like cell and of the tumor sample. As shown in Table 5 (first row), when shRNA-mediated knock-down data were used as reference, the ZEB2 activity index is significantly associated with the relative relapse risk in 6 out of 7 individual studies with balanced patient distribution, and when we used the pooled fRMA summarized data (Table 4). The increase in hazard ratio was also significant with ZEB2 activity index values categorized by quarters of their range or when the index was assigned a value of 1 or 0 whether the index values are respectively above (value=1) or below or equal to (value=0) an empirical threshold. This threshold is defined in detail in the section Material and Methods to the examples (Table 6). Next, we used an iterative leave-one-out approach to redefine the ZEB2 activity index in order to identify the shortest list of probe sets leading to an index that is best associated with relapse risk. We thereby selected a list of 16 probe sets (Table 7), corresponding to the shortest list of probe sets with the following characteristics:

i. provides an index value that is significantly associated with the risk of relapse in the pooled dataset;

ii. does so irrespectively of the way the ZEB2 activity index is expressed (raw data, data stratified in quarters or in dual categories);

iii. provides an index value that is significantly associated with the risk of relapse in most studies taken individually;

iv. provides an index value that is significantly associated with risk of relapse when different combinations of the individual studies are used to create the dataset; and

v. provides an index value that is stably associated with risk of relapse when it is cross-validated using the pooled data.

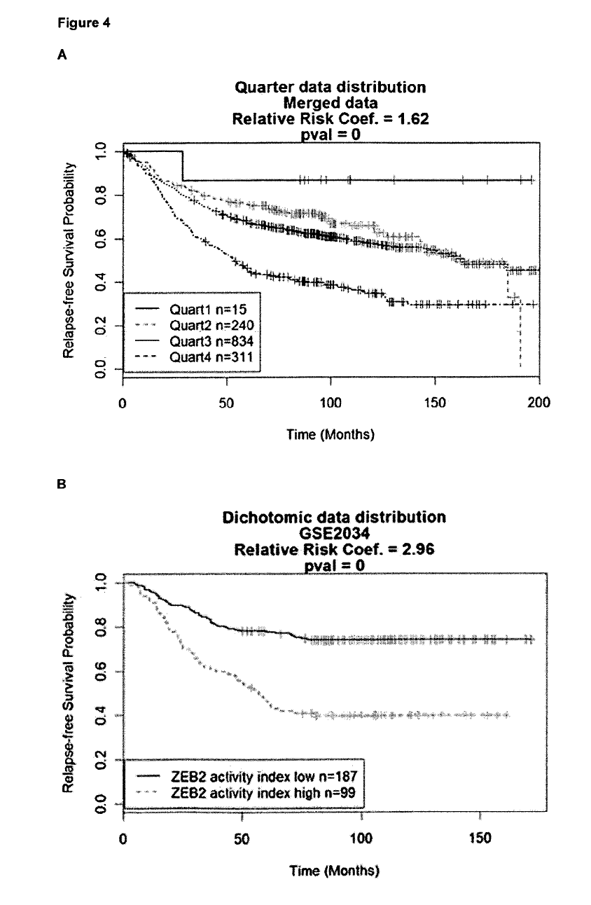

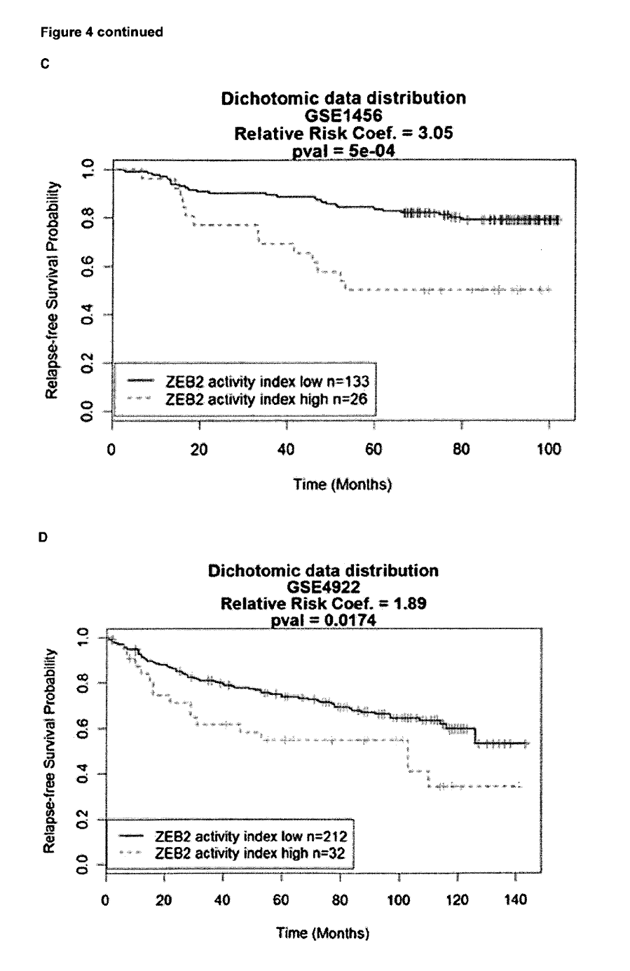

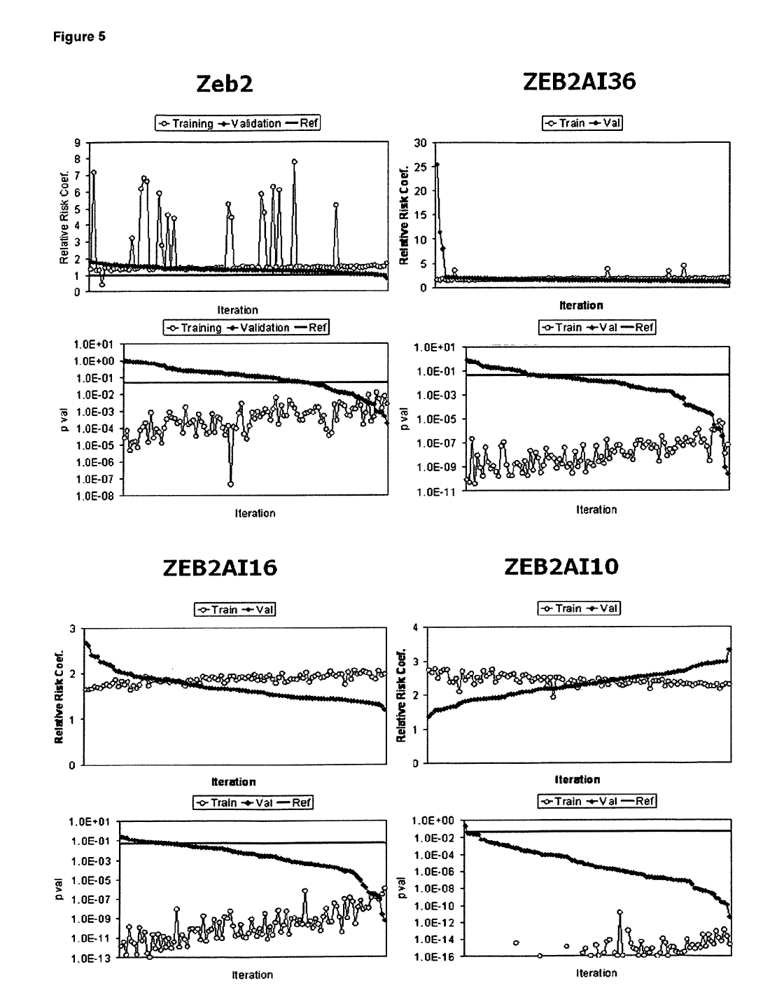

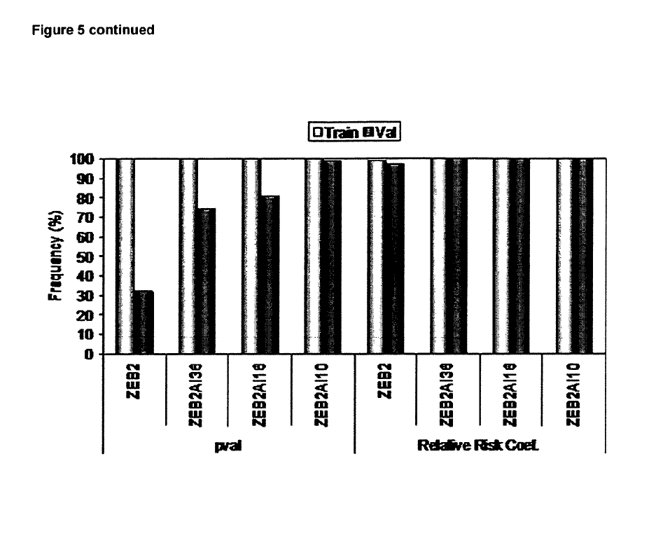

For the cross-validation analysis, patients from the pooled data set were randomly distributed 100 times into a training set composed of 75% (n=1050) of the samples and a complementary validation set consisting of the remaining samples (n=350) (FIG. 5). As illustrated in the Kaplan Meier curves in FIG. 4, the dichotomized ZEB2 activity index defined with these 16 core probe sets is significantly associated with an increased risk of relapse in the pooled dataset. This also holds true within studies and when pooled data are grouped in quarters of the ZEB2 index range. Finally, the relapse prediction on the basis of the dichotomic ZEB2 activity index values are accurate since only in 113 cases, no relapse was observed though the ZEB2 activity index was positive (false positive rate of 8.1% of total, 14% of cases without relapse).

TABLE 1

| Common Down | | |
|---|---|---|
| 39 probe sets | | |
| PROBE ID | HGNC | Gene ID |
| 202920_at | ANK2 | 287 |
| 206385_s_at | ANK3 | 288 |
| 219572_at | CADPS2 | 93664 |
| 211368_s_at | CASP1 | 834 |
| 200953_s_at | CCND2 | 894 |
| 201438_at | COL6A3 | 1293 |
| 219355_at | CXorf57 | 55086 |
| 204464_s_at | EDNRA | 1909 |
| 202669_s_at | EFNB2 | 1948 |
| 204643_s_at | ENOX2 | 10495 |
| 205278_at | GAD1 | 2571 |
| 203394_s_at | HES1 | 3280 |
| 203395_s_at | HES1 | 3280 |
| 205302_at | IGFBP1 | 3484 |
| 206693_at | IL7 | 3574 |
| 209098_s_at | JAG1 | 182 |
| 209099_x_at | JAG1 | 182 |
| 216268_s_at | JAG1 | 182 |
| 204734_at | KRT15 | 3866 |
| 202729_s_at | LTBP1 | 4052 |
| 203836_s_at | MAP3K5 | 4217 |
| 205442_at | MFAP3L | 9848 |
| 206022_at | NDP | 4693 |
| 205660_at | OASL | 8638 |
| 204134_at | PDE2A | 5138 |
| 210145_at | PLA2G4A | 5321 |
| 219483_s_at | PORCN | 64840 |
| 204337_at | RGS4 | 5999 |
| 203889_at | SCG5 | 6447 |
| 205421_at | SLC22A3 | 6581 |
| 204597_x_at | STC1 | 6781 |
| 219771_at | TBC1D8B | 54885 |
| 205513_at | TCN1 | 6947 |
| 203887_s_at | THBD | 7056 |
| 203888_at | THBD | 7056 |
| 221218_s_at | TPK1 | 27010 |
| 205844_at | VNN1 | 8876 |
| 206698_at | XK | 7504 |
| 203603_s_at | ZEB2 | 9839 |

TABLE 2

| | | | | Studies | | | | |
|---|---|---|---|---|---|---|---|---|
| | | | | GPL570 | | | GPL96 | |
| | | | | | GSE10890 | GSE12777 | E-TABM157 | GSE16795 |
| | | | | Charaffe | Adai | Januario | Neve | Holestelle |
| Lines | Tissue | Type | Intrinsic | Marseilles | Genentech | Genentech | UCSF | Rotterdam |
| BT-474 | b | Lu | Her | Char4 | GSM276004 | GSM320596 | Neve4 | GSM421861 |
| BT-483 | b | Lu | Lu | Char5 | GSM275979 | GSM320597 | Neve5 | GSM421862 |
| BT-549 | b | B | B | | GSM275974 | GSM320598 | Neve6 | GSM421863 |
| CAMA-1 | b | Lu | | Char6 | GSM276011 | GSM320599 | Neve7 | GSM421864 |
| DU4475 | b | B | | | GSM276006 | GSM320600 | Neve8 | GSM421865 |
| HCC1937 | b | B | A | Char8 | GSM275997 | GSM320621 | Neve21 | GSM421867 |
| HS578T | b | B | B | Char12 | GSM275977 | GSM320601 | Neve26 | GSM421868 |
| MCF7 | b | Lu | Lu | Char14 | GSM275978 | GSM320602 | Neve30 | GSM421869 |
| MDA-MB-175 | b | Lu | Lu | Char17 | GSM275976 | GSM320603 | Neve33 | GSM421872 |
| MDA-MB-231 | b | B | B | Char18 | GSM275993 | GSM320604 | Neve34 | GSM421873 |
| MDA-MB-361 | b | Lu | Her | | GSM275998 | GSM320605 | Neve35 | GSM421875 |
| MDA-MB-415 | b | Lu | Lu | | GSM276007 | GSM320606 | Neve36 | GSM421876 |
| MDA-MB-435s | b | B? | | | GSM275988 | GSM320607 | Neve37 | GSM421877 |
| MDA-MB-436 | b | B | B | | GSM275986 | GSM320608 | Neve38 | GSM421878 |
| MDA-MB-453 | b | Lu | Her | Char19 | GSM276003 | GSM320609 | Neve39 | GSM421879 |
| MDA-MB-468 | b | B | A | | GSM275994 | GSM320610 | Neve40 | GSM421880 |
| SKBR3 | b | Lu | Her | Char21 | GSM275983 | GSM320611 | Neve41 | GSM421883 |
| T47D | b | Lu | Lu | Char28 | GSM275991 | GSM320612 | Neve50 | GSM421894 |
| UACC-812 | b | Lu | Her | Char29 | GSM276032 | GSM320613 | Neve51 | GSM421895 |
| UACC-893 | b | Lu | Her | | GSM276033 | GSM320638 | | GSM421896 |
| ZR75-1 | b | Lu | Lu | Char30 | GSM276008 | GSM320614 | Neve52 | GSM421897 |

TABLE 3

| ID | | GSE12276 | GSE1456 | GSE2034 | GSE3494 | GSE4922 | GSE6532 | GSE6532 | GSE7390 | GSE9195 |
|---|---|---|---|---|---|---|---|---|---|---|
| Authors | | Bos | Pawitan | Wang | Miller | Ivshina | Loi2007 | Loi2007 | Desmedt | Loi2008 |
| Platform | | GPL570 | GPL96 | GPL96 | GPL96 | GPL96 | GPL570 | GPL96 | GPL96 | GPL570 |
| #Samples | | 204 | 159 | 286 | 251 | 289 | 87 | 327 | 198 | 77 |
| Scale | | Int | log | Int | log | log | log | log | log | log |
| Base log | | NA | e | NA | e | e | 2 | 2 | 2 | 2 |
| chemo | 0 | 167 | 0 | 0 | 0 | 0 | 0 | 0 | 0 | 0 |
| chemo | 1 | 37 | 0 | 0 | 0 | 0 | 0 | 0 | 0 | 0 |
| chemo | NA | 0 | 159 | 286 | 251 | 289 | 87 | 327 | 198 | 77 |
| e.dmfs | 0 | 57 | 0 | 196 | 0 | 0 | 59 | 225 | 136 | 67 |
| e.dmfs | 1 | 147 | 0 | 90 | 0 | 0 | 28 | 68 | 62 | 10 |
| e.dmfs | NA | 0 | 159 | 0 | 251 | 289 | 0 | 34 | 0 | 0 |
| e.rfs | 0 | 19 | 119 | 179 | 181 | 160 | 59 | 195 | 107 | 64 |
| e.rfs | 1 | 185 | 40 | 107 | 55 | 89 | 28 | 111 | 91 | 13 |
| e.rfs | NA | 0 | 0 | 0 | 15 | 40 | 0 | 21 | 0 | 0 |
| ER | 0 | 88 | 0 | 77 | 34 | 34 | 0 | 45 | 64 | 0 |
| ER | 1 | 116 | 0 | 209 | 213 | 211 | 0 | 264 | 134 | 0 |
| ER | NA | 0 | 159 | 0 | 4 | 44 | 87 | 18 | 0 | 77 |
| Grade | G0 | 15 | 0 | 0 | 0 | 0 | 0 | 0 | 0 | 0 |
| Grade | G1 | 65 | 28 | 89 | 67 | 68 | 17 | 68 | 30 | 14 |
| Grade | G2 | 2 | 58 | 7 | 128 | 166 | 37 | 143 | 83 | 20 |
| Grade | G3 | 122 | 61 | 190 | 54 | 55 | 16 | 64 | 83 | 24 |
| Grade | NA | 0 | 12 | 0 | 2 | 0 | 17 | 52 | 2 | 19 |
| Her | 0 | 166 | 0 | 240 | 0 | 0 | 0 | 0 | 0 | 0 |
| Her | 1 | 38 | 0 | 46 | 0 | 0 | 0 | 0 | 0 | 0 |
| Her | NA | 0 | 159 | 0 | 251 | 289 | 87 | 327 | 198 | 77 |
| Hormther | 0 | 166 | 0 | 0 | 0 | 0 | 0 | 0 | 0 | 0 |
| Hormther | 1 | 38 | 0 | 0 | 0 | 0 | 0 | 0 | 0 | 0 |
| Hormther | NA | 0 | 159 | 286 | 251 | 289 | 87 | 327 | 198 | 77 |
| Node | 0 | 0 | 0 | 0 | 158 | 159 | 29 | 221 | 0 | 41 |
| Node | 1 | 0 | 0 | 0 | 84 | 81 | 58 | 85 | 0 | 36 |
| Node | NA | 204 | 159 | 286 | 9 | 49 | 0 | 21 | 198 | 0 |
| p53 | 0 | 0 | 0 | 0 | 193 | 189 | 0 | 0 | 0 | 0 |
| p53 | 1 | 0 | 0 | 0 | 58 | 58 | 0 | 0 | 0 | 0 |
| p53 | NA | 204 | 159 | 286 | 193 | 42 | 87 | 327 | 198 | 77 |
| PR | 0 | 112 | 0 | 120 | 61 | 0 | 21 | 2 | 0 | 18 |
| PR | 1 | 92 | 0 | 166 | 190 | 0 | 64 | 46 | 0 | 59 |
| PR | NA | 0 | 159 | 0 | 61 | 289 | 0 | 279 | 198 | 0 |

TABLE 4

| | ZEB2 203603_s_at | | ZEB2AI36 | | ZEB2AI16 | |
|---|---|---|---|---|---|---|
| | Hazard | logrank | Hazard | logrank | Hazard | logrank |
| Study | ratio | p-value | ratio | p-value | ratio | p-value |
| Pooled data | 1.00 | 1.50E−01 | 24.49 | 5.13E−11 | 5.32 | 7.01E−12 |
| GSE12276 | 1.00 | 2.40E−01 | 0.94 | 9.33E−01 | 1.18 | 7.01E−01 |
| GSE1456 | 0.99 | 1.81E−01 | 29.87 | 6.93E−02 | 10.87 | 1.03E−02 |
| GSE2034 | 1.01 | 1.64E−01 | 90.66 | 1.88E−04 | 31.90 | 1.23E−06 |
| GSE3494 | 0.98 | 8.87E−02 | 37.73 | 4.36E−02 | 18.56 | 2.78E−03 |
| GSE4922 | 0.99 | 1.62E−01 | 24.92 | 2.33E−02 | 14.87 | 3.81E−04 |
| GSE6532g570 | 0.99 | 1.51E−01 | 73.89 | 2.32E−02 | 4.15 | 1.18E−01 |
| GSE6532g96 | 0.99 | 3.62E−01 | 20.97 | 9.13E−03 | 4.80 | 8.90E−03 |
| GSE7390 | 1.01 | 3.11E−01 | 293.03 | 2.09E−05 | 4.58 | 1.77E−02 |
| GSE9195 | 1.00 | 3.53E−01 | 41.86 | 2.78E−01 | 4.82 | 2.80E−01 |

TABLE 5

| | | GSE12276 | GSE1456 | GSE2034 | GSE3494 | GSE4922 |
|---|---|---|---|---|---|---|
| | | Baseline HR | | | | |
| ZEB2AI36 | | −6.31E−02 | 3.40E+00 | 4.51E+00 | 3.63E+00 | 3.22E+00 |
| ZEB2AI16 | | 1.69E−01 | 2.39E+00 | 3.46E+00 | 2.92E+00 | 2.70E+00 |
| 203603_s_at | ZEB2 | 1.56E−03 | −1.06E−02 | 1.00E−02 | −2.34E−02 | −1.36E−02 |
| 202920_at | ANK2 | −4.46E−04 | −7.78E−03 | −1.54E−03 | −6.23E−03 | −4.30E−03 |
| 206385_s_at | ANK3 | 1.76E−04 | −9.18E−04 | −4.16E−05 | −2.12E−04 | −3.46E−04 |
| 204643_s_at | ENOX2 | 2.60E−03 | −2.98E−03 | −1.41E−03 | 3.40E−03 | 6.53E−03 |
| 205278_at | GAD1 | −1.61E−04 | −9.58E−04 | −7.78E−04 | −1.83E−03 | −1.34E−03 |
| 203394_s_at | HES1 | 1.42E−04 | −1.12E−03 | −3.15E−04 | −3.72E−04 | −5.72E−04 |
| 204734_at | KRT15 | 1.82E−05 | −2.62E−04 | −1.37E−04 | −1.04E−04 | −8.33E−05 |
| 203836_s_at | MAP3K5 | 2.36E−04 | −2.49E−03 | −1.80E−03 | 6.85E−04 | 1.38E−03 |
| 205442_at | MFAP3L | 3.45E−04 | 1.16E−03 | 1.05E−03 | 2.36E−03 | 1.27E−03 |
| 205660_at | OASL | 2.17E−04 | 7.39E−04 | −1.16E−03 | 1.05E−03 | 1.81E−04 |
| 204134_at | PDE2A | −2.07E−03 | −8.81E−03 | −2.45E−03 | −5.28E−03 | −2.87E−03 |
| 204337_at | RGS4 | −6.34E−05 | −4.32E−04 | 1.79E−03 | 1.53E−03 | 1.54E−03 |
| 203889_at | SCG5 | 4.17E−04 | −6.51E−03 | 2.62E−04 | 6.02E−04 | 5.57E−04 |
| 204597_x_at | STC1 | −4.01E−05 | −5.32E−04 | 6.77E−05 | 2.97E−04 | 2.84E−04 |
| 219771_at | TBC1D8B | −7.91E−04 | −5.20E−03 | 6.65E−03 | −5.70E−03 | −4.99E−03 |
| 203887_s_at | THBD | 2.30E−04 | −3.81E−03 | −4.59E−04 | −2.39E−03 | −2.02E−03 |
| 219572_at | CADPS2 | −3.34E−05 | −1.96E−03 | −1.31E−04 | 4.22E−04 | −2.57E−04 |
| 200953_s_at | CCND2 | 6.07E−05 | −3.13E−03 | −6.05E−04 | −1.39E−03 | −6.91E−04 |
| 201438_at | COL6A3 | −1.72E−05 | −1.15E−04 | 9.03E−05 | −1.46E−04 | −6.67E−05 |
| 219355_at | CXorf57 | −7.17E−03 | −2.74E−02 | 1.37E−03 | −5.07E−03 | −5.74E−03 |
| 204464_s_at | EDNRA | −2.01E−04 | −1.60E−03 | 1.05E−03 | −1.31E−03 | −5.78E−04 |
| 202669_s_at | EFNB2 | 2.12E−04 | −1.90E−03 | −1.04E−03 | 1.27E−03 | 1.99E−04 |
| 205302_at | IGFBP1 | −5.56E−03 | −2.29E−03 | −6.88E−03 | 2.38E−02 | 4.80E−03 |
| 206693_at | IL7 | 9.47E−04 | −2.92E−02 | 9.17E−03 | −3.44E−03 | −3.93E−03 |
| 216268_s_at | JAG1 | 2.75E−04 | −1.31E−03 | 6.11E−04 | −1.01E−03 | −4.93E−04 |
| 209099_x_at | JAG1 | 2.61E−04 | −5.94E−04 | 7.78E−04 | −9.70E−04 | −4.61E−04 |
| 209098_s_at | JAG1 | 1.62E−03 | −3.11E−03 | 1.99E−03 | −1.22E−02 | −5.83E−03 |
| 202729_s_at | LTBP1 | 3.32E−04 | 5.17E−05 | −1.68E−04 | 5.45E−04 | 2.16E−04 |
| 206022_at | NDP | −5.38E−04 | 8.64E−04 | −3.13E−04 | −1.76E−03 | 2.24E−04 |
| 210145_at | PLA2G4A | 8.44E−04 | 2.23E−03 | −2.17E−03 | −2.24E−03 | 1.53E−03 |
| 219483_s_at | PORCN | −2.73E−03 | −7.56E−04 | 4.44E−04 | 5.68E−03 | 3.39E−03 |
| 205513_at | TCN1 | −9.02E−05 | 1.54E−05 | 6.64E−05 | 9.48E−05 | 1.72E−05 |
| 203888_at | THBD | 1.24E−04 | −6.09E−03 | −7.40E−03 | −8.55E−03 | −6.48E−03 |
| 221218_s_at | TPK1 | −5.56E−04 | −6.44E−03 | −2.86E−03 | −1.99E−03 | −7.24E−04 |
| 205844_at | VNN1 | 1.50E−03 | −1.33E−02 | −4.31E−03 | −4.09E−03 | −1.77E−03 |
| 206698_at | XK | 1.54E−03 | 2.04E−02 | 4.88E−03 | −1.63E−02 | −1.36E−02 |
| | | HR | | | | |
| ZEB2AI36 | | 9.39E−01 | 2.99E+01 | 9.07E+01 | 3.77E+01 | 2.49E+01 |
| ZEB2AI16 | | 1.18E+00 | 1.09E+01 | 3.19E+01 | 1.86E+01 | 1.49E+01 |
| 203603_s_at | ZEB2 | 1.00E+00 | 9.89E−01 | 1.01E+00 | 9.77E−01 | 9.87E−01 |
| 202920_at | ANK2 | 1.00E+00 | 9.92E−01 | 9.98E−01 | 9.94E−01 | 9.96E−01 |
| 206385_s_at | ANK3 | 1.00E+00 | 9.99E−01 | 1.00E+00 | 1.00E+00 | 1.00E+00 |
| 204643_s_at | ENOX2 | 1.00E+00 | 9.97E−01 | 9.99E−01 | 1.00E+00 | 1.01E+00 |
| 205278_at | GAD1 | 1.00E+00 | 9.99E−01 | 9.99E−01 | 9.98E−01 | 9.99E−01 |
| 203394_s_at | HES1 | 1.00E+00 | 9.99E−01 | 1.00E+00 | 1.00E+00 | 9.99E−01 |
| 204734_at | KRT15 | 1.00E+00 | 1.00E+00 | 1.00E+00 | 1.00E+00 | 1.00E+00 |
| 203836_s_at | MAP3K5 | 1.00E+00 | 9.98E−01 | 9.98E−01 | 1.00E+00 | 1.00E+00 |
| 205442_at | MFAP3L | 1.00E+00 | 1.00E+00 | 1.00E+00 | 1.00E+00 | 1.00E+00 |
| 205660_at | OASL | 1.00E+00 | 1.00E+00 | 9.99E−01 | 1.00E+00 | 1.00E+00 |
| 204134_at | PDE2A | 9.98E−01 | 9.91E−01 | 9.98E−01 | 9.95E−01 | 9.97E−01 |
| 204337_at | RGS4 | 1.00E+00 | 1.00E+00 | 1.00E+00 | 1.00E+00 | 1.00E+00 |
| 203889_at | SCG5 | 1.00E+00 | 9.94E−01 | 1.00E+00 | 1.00E+00 | 1.00E+00 |
| 204597_x_at | STC1 | 1.00E+00 | 9.99E−01 | 1.00E+00 | 1.00E+00 | 1.00E+00 |
| 219771_at | TBC1D8B | 9.99E−01 | 9.95E−01 | 1.01E+00 | 9.94E−01 | 9.95E−01 |
| 203887_s_at | THBD | 1.00E+00 | 9.96E−01 | 1.00E+00 | 9.98E−01 | 9.98E−01 |
| 219572_at | CADPS2 | 1.00E+00 | 9.98E−01 | 1.00E+00 | 1.00E+00 | 1.00E+00 |
| 200953_s_at | CCND2 | 1.00E+00 | 9.97E−01 | 9.99E−01 | 9.99E−01 | 9.99E−01 |
| 201438_at | COL6A3 | 1.00E+00 | 1.00E+00 | 1.00E+00 | 1.00E+00 | 1.00E+00 |
| 219355_at | CXorf57 | 9.93E−01 | 9.73E−01 | 1.00E+00 | 9.95E−01 | 9.94E−01 |
| 204464_s_at | EDNRA | 1.00E+00 | 9.98E−01 | 1.00E+00 | 9.99E−01 | 9.99E−01 |
| 202669_s_at | EFNB2 | 1.00E+00 | 9.98E−01 | 9.99E−01 | 1.00E+00 | 1.00E+00 |
| 205302_at | IGFBP1 | 9.94E−01 | 9.98E−01 | 9.93E−01 | 1.02E+00 | 1.00E+00 |
| 206693_at | IL7 | 1.00E+00 | 9.71E−01 | 1.01E+00 | 9.97E−01 | 9.96E−01 |
| 216268_s_at | JAG1 | 1.00E+00 | 9.99E−01 | 1.00E+00 | 9.99E−01 | 1.00E+00 |
| 209099_x_at | JAG1 | 1.00E+00 | 9.99E−01 | 1.00E+00 | 9.99E−01 | 1.00E+00 |
| 209098_s_at | JAG1 | 1.00E+00 | 9.97E−01 | 1.00E+00 | 9.88E−01 | 9.94E−01 |
| 202729_s_at | LTBP1 | 1.00E+00 | 1.00E+00 | 1.00E+00 | 1.00E+00 | 1.00E+00 |
| 206022_at | NDP | 9.99E−01 | 1.00E+00 | 1.00E+00 | 9.98E−01 | 1.00E+00 |
| 210145_at | PLA2G4A | 1.00E+00 | 1.00E+00 | 9.98E−01 | 9.98E−01 | 1.00E+00 |
| 219483_s_at | PORCN | 9.97E−01 | 9.99E−01 | 1.00E+00 | 1.01E+00 | 1.00E+00 |
| 205513_at | TCN1 | 1.00E+00 | 1.00E+00 | 1.00E+00 | 1.00E+00 | 1.00E+00 |
| 203888_at | THBD | 1.00E+00 | 9.94E−01 | 9.93E−01 | 9.91E−01 | 9.94E−01 |
| 221218_s_at | TPK1 | 9.99E−01 | 9.94E−01 | 9.97E−01 | 9.98E−01 | 9.99E−01 |
| 205844_at | VNN1 | 1.00E+00 | 9.87E−01 | 9.96E−01 | 9.96E−01 | 9.98E−01 |
| 206698_at | XK | 1.00E+00 | 1.02E+00 | 1.00E+00 | 9.84E−01 | 9.86E−01 |
| | | logrankp | | | | |
| ZEB2AI36 | | 9.33E−01 | 6.93E−02 | 1.88E−04 | 4.36E−02 | 2.33E−02 |
| ZEB2AI16 | | 7.01E−01 | 1.03E−02 | 1.23E−06 | 2.78E−03 | 3.81E−04 |
| 203603_s_at | ZEB2 | 2.40E−01 | 1.81E−01 | 1.64E−01 | 8.87E−02 | 1.62E−01 |
| 202920_at | ANK2 | 4.99E−01 | 1.34E−02 | 2.85E−01 | 3.85E−02 | 4.24E−02 |
| 206385_s_at | ANK3 | 1.57E−01 | 2.02E−01 | 8.76E−01 | 7.14E−01 | 4.71E−01 |
| 204643_s_at | ENOX2 | 2.29E−03 | 5.11E−01 | 3.78E−01 | 2.49E−01 | 7.73E−03 |
| 205278_at | GAD1 | 7.31E−01 | 6.98E−01 | 6.36E−01 | 2.01E−01 | 1.86E−01 |
| 203394_s_at | HES1 | 6.32E−01 | 3.24E−01 | 3.46E−01 | 4.20E−01 | 1.25E−01 |
| 204734_at | KRT15 | 8.29E−01 | 1.67E−01 | 8.91E−02 | 2.58E−01 | 2.14E−01 |
| 203836_s_at | MAP3K5 | 5.27E−01 | 1.83E−01 | 2.47E−01 | 4.98E−01 | 1.29E−01 |
| 205442_at | MFAP3L | 2.86E−01 | 4.99E−01 | 2.65E−01 | 3.94E−02 | 2.62E−01 |
| 205660_at | OASL | 5.48E−01 | 3.39E−01 | 1.05E−01 | 1.97E−01 | 8.16E−01 |
| 204134_at | PDE2A | 2.97E−01 | 8.41E−03 | 2.97E−01 | 1.63E−02 | 4.35E−02 |
| 204337_at | RGS4 | 8.81E−01 | 7.91E−01 | 1.37E−03 | 4.01E−02 | 2.48E−02 |
| 203889_at | SCG5 | 4.01E−01 | 3.71E−01 | 1.17E−02 | 4.22E−03 | 3.86E−03 |
| 204597_x_at | STC1 | 8.74E−01 | 3.87E−01 | 6.92E−01 | 2.12E−01 | 1.39E−01 |
| 219771_at | TBC1D8B | 4.71E−01 | 2.62E−01 | 6.12E−02 | 2.75E−01 | 2.15E−01 |
| 203887_s_at | THBD | 6.06E−01 | 7.90E−02 | 6.29E−01 | 8.54E−02 | 5.26E−02 |
| 219572_at | CADPS2 | 9.47E−01 | 8.24E−02 | 8.28E−01 | 6.42E−01 | 7.32E−01 |
| 200953_s_at | CCND2 | 7.63E−01 | 1.87E−04 | 1.82E−01 | 2.99E−02 | 1.49E−01 |
| 201438_at | COL6A3 | 6.18E−01 | 5.22E−02 | 5.00E−02 | 3.77E−03 | 9.44E−02 |
| 219355_at | CXorf57 | 8.21E−02 | 1.52E−01 | 7.79E−01 | 6.30E−01 | 4.92E−01 |
| 204464_s_at | EDNRA | 4.23E−01 | 3.17E−02 | 6.08E−03 | 5.25E−02 | 2.43E−01 |
| 202669_s_at | EFNB2 | 3.70E−01 | 2.82E−02 | 6.18E−01 | 1.69E−01 | 8.19E−01 |
| 205302_at | IGFBP1 | 2.10E−01 | 9.60E−01 | 6.48E−01 | 5.05E−01 | 8.58E−01 |
| 206693_at | IL7 | 4.53E−01 | 3.00E−01 | 5.60E−01 | 8.74E−01 | 8.10E−01 |
| 216268_s_at | JAG1 | 1.56E−01 | 1.61E−01 | 5.77E−02 | 1.16E−01 | 3.15E−01 |
| 209099_x_at | JAG1 | 1.94E−01 | 3.19E−01 | 2.39E−03 | 3.65E−02 | 1.69E−01 |
| 209098_s_at | JAG1 | 6.01E−02 | 4.15E−01 | 5.55E−01 | 3.25E−02 | 1.48E−01 |
| 202729_s_at | LTBP1 | 1.65E−01 | 9.40E−01 | 6.64E−01 | 4.23E−01 | 6.79E−01 |
| 206022_at | NDP | 1.77E−01 | 4.97E−01 | 5.58E−01 | 2.45E−01 | 6.17E−01 |
| 210145_at | PLA2G4A | 5.69E−04 | 3.00E−01 | 5.37E−01 | 7.52E−01 | 1.35E−01 |
| 219483_s_at | PORCN | 2.73E−01 | 8.76E−01 | 8.74E−01 | 6.78E−02 | 2.64E−01 |
| 205513_at | TCN1 | 4.01E−01 | 8.93E−01 | 3.03E−01 | 2.42E−01 | 8.25E−01 |
| 203888_at | THBD | 8.86E−01 | 1.86E−01 | 3.63E−02 | 3.47E−02 | 2.81E−02 |
| 221218_s_at | TPK1 | 5.59E−01 | 4.73E−01 | 3.20E−01 | 6.03E−01 | 6.71E−01 |
| 205844_at | VNN1 | 4.99E−01 | 6.17E−01 | 2.66E−01 | 7.41E−01 | 7.12E−01 |
| 206698_at | XK | 2.33E−01 | 5.07E−05 | 1.60E−01 | 2.49E−01 | 2.10E−01 |

| | | GSE6532g570 | GSE6532g96 | GSE7390 | GSE9195 |
|---|---|---|---|---|---|
| | | Baseline HR | | | |
| ZEB2AI36 | | 4.30E+00 | 3.04E+00 | 5.68E+00 | 3.73E+00 |
| ZEB2AI16 | | 1.42E+00 | 1.57E+00 | 1.52E+00 | 1.57E+00 |
| 203603_s_at | ZEB2 | −5.04E−03 | −6.31E−03 | 9.58E−03 | −4.81E−03 |
| 202920_at | ANK2 | −2.41E−03 | −3.53E−03 | 4.46E−04 | −3.09E−03 |
| 206385_s_at | ANK3 | −1.16E−04 | −4.70E−05 | −1.08E−03 | −4.21E−04 |
| 204643_s_at | ENOX2 | −8.66E−04 | 1.01E−03 | −3.94E−03 | −1.56E−02 |
| 205278_at | GAD1 | 3.30E−04 | −5.28E−04 | −3.69E−02 | −6.19E−04 |
| 203394_s_at | HES1 | −5.87E−05 | 8.34E−05 | 5.65E−04 | 1.21E−04 |
| 204734_at | KRT15 | 2.45E−05 | −1.02E−05 | −7.22E−05 | −1.58E−03 |
| 203836_s_at | MAP3K5 | 4.10E−04 | 4.60E−04 | −2.87E−03 | −9.09E−04 |
| 205442_at | MFAP3L | −5.50E−04 | 3.92E−04 | 6.10E−04 | −7.83E−03 |
| 205660_at | OASL | −2.35E−04 | 9.18E−04 | −2.80E−04 | 1.35E−03 |
| 204134_at | PDE2A | −4.26E−03 | −2.03E−03 | 2.19E−03 | −1.10E−02 |
| 204337_at | RGS4 | 5.38E−04 | 1.42E−03 | 3.56E−04 | −1.46E−03 |
| 203889_at | SCG5 | 2.47E−05 | 2.88E−04 | −1.04E−04 | 3.41E−04 |
| 204597_x_at | STC1 | 7.13E−05 | 3.51E−04 | 6.67E−04 | −7.73E−05 |
| 219771_at | TBC1D8B | −1.49E−03 | 2.19E−04 | −1.12E−03 | −9.80E−03 |
| 203887_s_at | THBD | −1.43E−03 | −1.66E−03 | −6.61E−04 | −1.79E−03 |
| 219572_at | CADPS2 | −1.91E−03 | −2.92E−04 | −8.52E−04 | −2.99E−03 |
| 200953_s_at | CCND2 | −5.44E−04 | −1.01E−03 | −4.17E−04 | 1.46E−03 |
| 201438_at | COL6A3 | −1.15E−04 | −1.13E−05 | 6.06E−05 | −2.04E−05 |
| 219355_at | CXorf57 | 1.16E−03 | 2.54E−03 | −1.01E−02 | −3.27E−02 |
| 204464_s_at | EDNRA | 2.19E−04 | −1.34E−04 | 1.20E−04 | 1.44E−04 |
| 202669_s_at | EFNB2 | 2.62E−03 | 8.98E−04 | 3.36E−05 | 2.97E−03 |
| 205302_at | IGFBP1 | 6.25E−03 | −1.90E−03 | −7.20E−06 | −1.45E−02 |
| 206693_at | IL7 | 1.30E−02 | −6.10E−03 | 5.98E−03 | 7.22E−02 |
| 216268_s_at | JAG1 | 4.86E−04 | 7.47E−05 | 8.80E−04 | −4.10E−04 |
| 209099_x_at | JAG1 | 4.71E−04 | −1.62E−04 | 5.53E−04 | −7.68E−04 |
| 209098_s_at | JAG1 | 5.22E−03 | 1.47E−04 | 8.52E−03 | −6.42E−03 |
| 202729_s_at | LTBP1 | 5.96E−04 | 3.40E−04 | 2.19E−04 | 1.06E−03 |
| 206022_at | NDP | 7.68E−06 | 5.23E−04 | 4.15E−04 | −7.62E−05 |
| 210145_at | PLA2G4A | −1.67E−02 | −8.66E−03 | −1.44E−03 | 3.04E−03 |
| 219483_s_at | PORCN | 1.10E−02 | 1.43E−03 | 3.25E−03 | −2.51E−03 |
| 205513_at | TCN1 | −8.85E−05 | 4.52E−05 | −1.04E−04 | 2.75E−04 |
| 203888_at | THBD | −2.69E−03 | −5.11E−03 | −2.80E−03 | −4.58E−03 |
| 221218_s_at | TPK1 | −6.79E−03 | −6.17E−03 | 1.25E−03 | −1.61E−04 |
| 205844_at | VNN1 | −3.24E−03 | −7.19E−03 | 1.03E−02 | −9.59E−02 |
| 206698_at | XK | −1.42E−04 | 7.75E−05 | 4.31E−03 | 4.41E−03 |
| | | HR | | | |
| ZEB2AI36 | | 7.39E+01 | 2.10E+01 | 2.93E+02 | 4.19E+01 |
| ZEB2AI16 | | 4.15E+00 | 4.80E+00 | 4.58E+00 | 4.82E+00 |
| 203603_s_at | ZEB2 | 9.95E−01 | 9.94E−01 | 1.01E+00 | 9.95E−01 |
| 202920_at | ANK2 | 9.98E−01 | 9.96E−01 | 1.00E+00 | 9.97E−01 |
| 206385_s_at | ANK3 | 1.00E+00 | 1.00E+00 | 9.99E−01 | 1.00E+00 |
| 204643_s_at | ENOX2 | 9.99E−01 | 1.00E+00 | 9.96E−01 | 9.85E−01 |
| 205278_at | GAD1 | 1.00E+00 | 9.99E−01 | 9.64E−01 | 9.99E−01 |
| 203394_s_at | HES1 | 1.00E+00 | 1.00E+00 | 1.00E+00 | 1.00E+00 |
| 204734_at | KRT15 | 1.00E+00 | 1.00E+00 | 1.00E+00 | 9.98E−01 |
| 203836_s_at | MAP3K5 | 1.00E+00 | 1.00E+00 | 9.97E−01 | 9.99E−01 |
| 205442_at | MFAP3L | 9.99E−01 | 1.00E+00 | 1.00E+00 | 9.92E−01 |
| 205660_at | OASL | 1.00E+00 | 1.00E+00 | 1.00E+00 | 1.00E+00 |
| 204134_at | PDE2A | 9.96E−01 | 9.98E−01 | 1.00E+00 | 9.89E−01 |
| 204337_at | RGS4 | 1.00E+00 | 1.00E+00 | 1.00E+00 | 9.99E−01 |
| 203889_at | SCG5 | 1.00E+00 | 1.00E+00 | 1.00E+00 | 1.00E+00 |
| 204597_x_at | STC1 | 1.00E+00 | 1.00E+00 | 1.00E+00 | 1.00E+00 |
| 219771_at | TBC1D8B | 9.99E−01 | 1.00E+00 | 9.99E−01 | 9.90E−01 |
| 203887_s_at | THBD | 9.99E−01 | 9.98E−01 | 9.99E−01 | 9.98E−01 |
| 219572_at | CADPS2 | 9.98E−01 | 1.00E+00 | 9.99E−01 | 9.97E−01 |
| 200953_s_at | CCND2 | 9.99E−01 | 9.99E−01 | 1.00E+00 | 1.00E+00 |
| 201438_at | COL6A3 | 1.00E+00 | 1.00E+00 | 1.00E+00 | 1.00E+00 |
| 219355_at | CXorf57 | 1.00E+00 | 1.00E+00 | 9.90E−01 | 9.68E−01 |
| 204464_s_at | EDNRA | 1.00E+00 | 1.00E+00 | 1.00E+00 | 1.00E+00 |
| 202669_s_at | EFNB2 | 1.00E+00 | 1.00E+00 | 1.00E+00 | 1.00E+00 |
| 205302_at | IGFBP1 | 1.01E+00 | 9.98E−01 | 1.00E+00 | 9.86E−01 |
| 206693_at | IL7 | 1.01E+00 | 9.94E−01 | 1.01E+00 | 1.07E+00 |
| 216268_s_at | JAG1 | 1.00E+00 | 1.00E+00 | 1.00E+00 | 1.00E+00 |
| 209099_x_at | JAG1 | 1.00E+00 | 1.00E+00 | 1.00E+00 | 9.99E−01 |
| 209098_s_at | JAG1 | 1.01E+00 | 1.00E+00 | 1.01E+00 | 9.94E−01 |
| 202729_s_at | LTBP1 | 1.00E+00 | 1.00E+00 | 1.00E+00 | 1.00E+00 |
| 206022_at | NDP | 1.00E+00 | 1.00E+00 | 1.00E+00 | 1.00E+00 |
| 210145_at | PLA2G4A | 9.83E−01 | 9.91E−01 | 9.99E−01 | 1.00E+00 |
| 219483_s_at | PORCN | 1.01E+00 | 1.00E+00 | 1.00E+00 | 9.97E−07 |
| 205513_at | TCN1 | 1.00E+00 | 1.00E+00 | 1.00E+00 | 1.00E+00 |
| 203888_at | THBD | 9.97E−01 | 9.95E−01 | 9.97E−01 | 9.95E−01 |
| 221218_s_at | TPK1 | 9.93E−01 | 9.94E−01 | 1.00E+00 | 1.00E+00 |
| 205844_at | VNN1 | 9.97E−01 | 9.93E−01 | 1.01E+00 | 9.09E−01 |
| 206698_at | XK | 1.00E+00 | 1.00E+00 | 1.00E+00 | 1.00E+00 |
| | | logrankp | | | |
| ZEB2AI36 | | 2.32E−02 | 9.13E−03 | 2.09E−05 | 2.78E−01 |
| ZEB2AI16 | | 1.18E−01 | 8.90E−03 | 1.77E−02 | 2.80E−01 |
| 203603_s_at | ZEB2 | 1.51E−01 | 3.62E−01 | 3.11E−01 | 3.53E−01 |
| 202920_at | ANK2 | 1.97E−01 | 4.94E−02 | 7.64E−01 | 5.13E−01 |
| 206385_s_at | ANK3 | 7.67E−01 | 8.93E−01 | 4.35E−03 | 6.14E−01 |
| 204643_s_at | ENOX2 | 8.20E−01 | 6.02E−01 | 1.28E−01 | 5.73E−02 |
| 205278_at | GAD1 | 7.31E−01 | 5.18E−01 | 1.01E−01 | 6.86E−01 |
| 203394_s_at | HES1 | 9.16E−01 | 7.80E−01 | 2.66E−01 | 7.95E−01 |
| 204734_at | KRT15 | 8.47E−01 | 8.93E−01 | 4.35E−01 | 1.62E−01 |
| 203836_s_at | MAP3K5 | 7.73E−01 | 6.45E−01 | 1.16E−02 | 7.60E−01 |
| 205442_at | MFAP3L | 5.84E−01 | 5.30E−01 | 3.28E−01 | 1.48E−01 |
| 205660_at | OASL | 8.39E−01 | 1.57E−01 | 6.15E−01 | 2.17E−01 |
| 204134_at | PDE2A | 4.54E−01 | 1.42E−01 | 4.04E−01 | 1.88E−01 |
| 204337_at | RGS4 | 4.62E−01 | 3.29E−02 | 1.39E−01 | 5.00E−01 |
| 203889_at | SCG5 | 9.76E−01 | 2.28E−01 | 7.05E−01 | 1.54E−02 |
| 204597_x_at | STC1 | 8.88E−01 | 1.93E−01 | 2.69E−01 | 8.80E−01 |
| 219771_at | TBC1D8B | 5.43E−01 | 9.39E−01 | 5.50E−01 | 1.01E−01 |
| 203887_s_at | THBD | 1.90E−01 | 7.92E−02 | 5.13E−01 | 2.73E−01 |
| 219572_at | CADPS2 | 7.24E−02 | 5.05E−01 | 2.64E−01 | 8.95E−02 |
| 200953_s_at | CCND2 | 4.15E−01 | 2.82E−02 | 2.95E−01 | 3.54E−01 |
| 201438_at | COL6A3 | 2.57E−01 | 7.44E−01 | 1.83E−01 | 8.78E−01 |
| 219355_at | CXorf57 | 8.45E−01 | 6.93E−01 | 1.77E−01 | 1.71E−01 |
| 204464_s_at | EDNRA | 7.18E−01 | 7.37E−01 | 7.47E−01 | 8.86E−01 |
| 202669_s_at | EFNB2 | 1.06E−01 | 2.07E−01 | 9.19E−01 | 7.30E−02 |
| 205302_at | IGFBP1 | 1.22E−01 | 8.90E−01 | 1.00E+00 | 5.97E−01 |
| 206693_at | IL7 | 4.06E−02 | 7.97E−01 | 6.88E−01 | 7.82E−02 |
| 216268_s_at | JAG1 | 4.14E−01 | 8.35E−01 | 4.57E−03 | 7.30E−01 |
| 209099_x_at | JAG1 | 4.54E−01 | 5.96E−01 | 7.77E−02 | 5.83E−01 |
| 209098_s_at | JAG1 | 3.18E−01 | 9.64E−01 | 2.08E−03 | 5.64E−01 |
| 202729_s_at | LTBP1 | 2.80E−01 | 4.04E−01 | 5.53E−01 | 1.92E−01 |
| 206022_at | NDP | 9.79E−01 | 8.33E−02 | 5.07E−01 | 9.05E−01 |
| 210145_at | PLA2G4A | 9.39E−02 | 3.39E−01 | 3.54E−01 | 7.32E−01 |
| 219483_s_at | PORCN | 1.11E−01 | 5.49E−01 | 2.84E−01 | 7.41E−01 |
| 205513_at | TCN1 | 6.11E−01 | 2.83E−01 | 1.83E−01 | 4.77E−01 |
| 203888_at | THBD | 2.37E−01 | 3.27E−02 | 3.00E−01 | 3.10E−01 |
| 221218_s_at | TPK1 | 1.28E−01 | 1.37E−01 | 6.01E−01 | 9.76E−01 |
| 205844_at | VNN1 | 7.50E−01 | 5.40E−01 | 4.85E−03 | 3.08E−01 |
| 206698_at | XK | 9.71E−01 | 9.87E−01 | 4.34E−01 | 4.31E−02 |

TABLE 6

| | | Hazard Ratio | logrankp | Lconfint | Hconfint | zpval |
|---|---|---|---|---|---|---|
| Raw | ZEB2AI36 | 24.5 | 5.1E−11 | 9.4 | 63.6 | 0.95 |
| | ZEB2AI16 | 5.3 | 7.0E−12 | 3.3 | 8.6 | 0.34 |
| quarters | ZEB2AI36 | 1.5 | 5.9E−10 | 1.3 | 1.7 | 0.59 |
| | ZEB2AI16 | 1.6 | 1.2E−13 | 1.4 | 1.8 | 0.69 |
| Treshold | ZEB2AI36 | 1.6 | 3.5E−09 | 1.4 | 1.9 | 0.60 |
| | ZEB2AI16 | 2.0 | 6.1E−15 | 1.7 | 2.3 | 0.38 |

TABLE 7

| Probes | HUGO names |
|---|---|
| 203603_s_at | ZEB2 |
| 202920_at | ANK2 |
| 206385_s_at | ANK3 |
| 204643_s_at | ENOX2 |
| 205278_at | GAD1 |
| 203394_s_at | HES1 |
| 204734_at | KRT15 |
| 203836_s_at | MAP3K5 |
| 205442_at | MFAP3L |
| 205660_at | OASL |
| 204134_at | PDE2A |
| 204337_at | RGS4 |
| 203889_at | SCG5 |
| 204597_x_at | STC1 |
| 219771_at | TBC1D8B |
| 203887_s_at | THBD |

TABLE 8

| Lines | Tissue | Type | Intrinsic |
|---|---|---|---|
| 184B5 | b | B | B |
| BT-549 | b | B | B |
| HBL100 | b | B | B |
| HCC1395 | b | B | B |
| HCC1806 | b | B | B |
| HCC38 | b | B | B |
| HDQ-P1 | b | B | B |
| HME-1 | b | B | B |
| HS578T | b | B | B |
| MCF10A | b | B | B |
| MCF12A | b | B | B |
| MDA-MB-231 | b | B | B |
| MDA-MB-436 | b | B | B |
| SK-BR-7 | b | B | B |
| SUM1315MO2 | b | B | B |
| SUM149PT | b | B | B |
| SUM159PT | b | B | B |
| SUM-225 | b | B | B |
| SW527 | b | B | B |
| CAL-120 | b | B | |
| CAL-148 | b | B | |
| CAL-51 | b | B | |
| CAL85-1 | b | B | |
| DU4475 | b | B | |
| MDA-MB-435s | b | B | |
| SUM102PT | b | B | |
| SUM229PE | b | B | |
| BT-20 | b | B | A |
| HCC1143 | b | B | A |
| HCC1187 | b | B | A |
| HCC1569 | b | B | A |
| HCC1599 | b | B | A |
| HCC1937 | b | B | A |
| HCC1954 | b | B | A |
| HCC2157 | b | B | A |
| HCC3153 | b | B | A |
| HCC70 | b | B | A |
| MDA-MB-468 | b | B | A |
| AU-565 | b | Lu | Her |
| BT-474 | b | Lu | Her |
| EFM-192A | b | Lu | Her |
| EVSA-T | b | Lu | Her |
| HCC1419 | b | Lu | Her |
| JIMT-1 | b | Lu | Her |
| KPL4 | b | Lu | Her |
| MDA-MB-330 | b | Lu | Her |
| MDA-MB-361 | b | Lu | Her |
| MDA-MB-453 | b | Lu | Her |
| SKBR3 | b | Lu | Her |
| SUM-190 | b | Lu | Her |
| UACC-812 | b | Lu | Her |
| UACC-893 | b | Lu | Her |
| 600MPE | b | Lu | Lu |
| BT-483 | b | Lu | Lu |
| HCC1428 | b | Lu | Lu |
| HCC1500 | b | Lu | Lu |
| HCC202 | b | Lu | Lu |
| HCC2185 | b | Lu | Lu |
| HCC2218 | b | Lu | Lu |
| KPL1 | b | Lu | Lu |
| LY2 | b | Lu | Lu |
| MCF7 | b | Lu | Lu |
| MDA-MB-134 | b | Lu | Lu |
| VI | | | |
| MDA-MB-157 | b | Lu | Lu |
| MDA-MB-175 | b | Lu | Lu |
| MDA-MB-415 | b | Lu | Lu |
| MFM-223 | b | Lu | Lu |
| MPE600 | b | Lu | Lu |
| OCUB-F | b | Lu | Lu |
| OCUB-M | | Lu | Lu |
| SK-BR-5 | b | Lu | Lu |
| SUM44PE | b | Lu | Lu |
| SUM-52 | b | Lu | Lu |
| T47D | b | Lu | Lu |
| ZR75-1 | b | Lu | Lu |
| ZR-75-30 | b | Lu | Lu |
| ZR75B | b | Lu | Lu |
| BrCa-MZ-01 | b | ND | |
| BT474.tb | b | | |
| BT474EI | b | | |
| BT5491 | b | | |
| CAL1201 | b | | |
| CAMA-1 | b | Lu | |
| EFM19 | b | Lu | |
| EVSA1 | b | | |
| H3396 | b | | |
| HCC1007 | b | Lu | |
| HCC1008 | b | | |
| MCF7/Her2 | b | | |
| MDAMB361.1 | b | | |
| MDA-N | b | | |
| MX1 | b | | |
| S68 | b | | |
| SKBR31 | b | | |
| SUM-185 | b | Lu | |
| T47D1 | b | | |

TABLE 9

| | | Delta |
|---|---|---|
| ProbeID | Hugo | WT/ZEB2KO |
| 203603_s_at | ZEB2 | 46.67860711 |
| 202920_at | ANK2 | 52.18657981 |
| 206385_s_at | ANK3 | 67.03719494 |
| 219572_at | CADPS2 | 44.56233457 |
| 200953_s_at | CCND2 | 121.6439492 |
| 201438_at | COL6A3 | 119.2486347 |
| 219355_at | CXorf57 | 78.87993464 |
| 204464_s_at | EDNRA | 54.19869654 |
| 202669_s_at | EFNB2 | 135.0398204 |
| 204643_s_at | ENOX2 | 178.883559 |
| 205278_at | GAD1 | 41.51629258 |
| 203394_s_at | HES1 | 125.3137015 |
| 205302_at | IGFBP1 | 328.3477944 |
| 206693_at | IL7 | 27.83808197 |
| 216268_s_at | JAG1 | 1220.62335 |
| 209099_x_at | JAG1 | 882.4718998 |
| 209098_s_at | JAG1 | 299.8234846 |
| 204734_at | KRT15 | 123.4843941 |
| 202729_s_at | LTBP1 | 174.6522584 |
| 203836_s_at | MAP3K5 | 87.22793593 |
| 205442_at | MFAP3L | 128.99717 |
| 206022_at | NDP | 100.9133248 |
| 205660_at | OASL | 163.3662504 |
| 204134_at | PDE2A | 52.66818863 |
| 210145_at | PLA2G4A | 21.99697453 |
| 219483_s_at | PORCN | 114.006106 |
| 204337_at | RGS4 | 668.3911874 |
| 203889_at | SCG5 | 517.3432789 |
| 204597_x_at | STC1 | 343.1721409 |
| 219771_at | TBC1D8B | 61.08020634 |
| 205513_at | TCN1 | 41.43771857 |
| 203888_at | THBD | 113.3828307 |
| 203887_s_at | THBD | 332.9581919 |
| 221218_s_at | TPK1 | 83.3430321 |
| 205844_at | VNN1 | 52.61176289 |
| 206698_at | XK | 125.914677 |

TABLE 10

| | Total | Select1 | Select2 | Select3 | Select4 | Select5 | Select6 | Select7 |
|---|---|---|---|---|---|---|---|---|
| GSE12276 | 204 | 204 | 0 | 0 | 0 | 204 | 204 | 204 |
| GSE1456 | 159 | 159 | 159 | 159 | 159 | 159 | 159 | 159 |
| GSE2034 | 286 | 286 | 286 | 286 | 286 | 286 | 286 | 286 |
| GSE3494 | 251 | 2 | 2 | 2 | 2 | 2 | 2 | 2 |
| GSE4922 | 289 | 244 | 249 | 289 | 249 | 289 | 249 | 249 |
| GSE6532g570 | 87 | 86 | 0 | 0 | 0 | 87 | 0 | 87 |
| GSE6532g96 | 327 | 174 | 9 | 187 | 9 | 187 | 9 | 9 |
| GSE7390 | 198 | 168 | 32 | 179 | 32 | 179 | 32 | 32 |
| GSE9195 | 77 | 77 | 0 | 0 | 77 | 77 | 77 | 77 |
| Total | 1878 | 1400 | 737 | 1102 | 814 | 1470 | 1018 | 1105 |

TABLE 11

| | Select1 | Select2 |
|---|---|---|
| GSE12276 | Full | Removed (GPL570) |
| GSE1456 | Full | Full |
| GSE2034 | Full | Full |
| GSE3494 | Removed redundant with 4922 | Removed redundant with 4922 |
| GSE4922 | Removed samples without time value | Removed samples without time value |
| GSE6532g570 | Removed samples with time value >200 | Removed (GPL570) |
| GSE6532g96 | Removed redundant with 4922 | Removed redundant with 4922 |
| | Removed samples with time value >200 | Removed samples not analysed in Karolinska |
| GSE7390 | Removed redundant with 4922 | Removed redundant with 4922 |
| | Removed samples with time value >200 | Removed samples not analysed in Karolinska |
| GSE9195 | Full | Removed (GPL570) |

| | Select3 | Select4 |
|---|---|---|
| GSE12276 | Removed (GPL570) | Removed |
| GSE1456 | Full | Full |
| GSE2034 | Full | Full |
| GSE3494 | Removed redundant with 4922 | Removed redundant with 4922 |
| GSE4922 | Full | Removed samples without time value |
| GSE6532g570 | Removed (GPL570) | Removed |
| GSE6532g96 | Removed redundant with 4922 | Removed redundant with 4922 |
| | | Removed samples not analysed in Karolinska |
| GSE7390 | Removed redundant with 4922 | Removed redundant with 4922 |
| | | Removed samples not analysed in Karolinska |
| GSE9195 | Removed (GPL570) | Full |
| | Select5 | Select6 |
| GSE12276 | Full | Full |
| GSE1456 | Full | Full |
| GSE2034 | Full | Full |
| GSE3494 | Removed redundant with 4922 | Removed redundant with 4922 |
| GSE4922 | Full | Removed samples without time value |
| GSE6532g570 | Full | Removed |
| GSE6532g96 | Removed redundant with 4922 | Removed redundant with 4922 |
| | | Removed samples not analysed in Karolinska |
| GSE7390 | Removed redundant with 4922 | Removed redundant with 4922 |
| | | Removed samples not analysed in Karolinska |
| GSE9195 | Full | Full |

| | Select7 |
|---|---|
| GSE12276 | Full |
| GSE1456 | Full |
| GSE2034 | Full |
| GSE3494 | Removed redundant with 4922 |
| GSE4922 | Removed samples without time value |
| GSE6532g570 | Full |
| GSE6532g96 | Removed redundant with 4922 |
| | Removed samples not analysed in Karolinska |
| GSE7390 | Removed redundant with 4922 |
| | Removed samples not analysed in Karolinska |
| GSE9195 | Full |

TABLE 12

| | # probesets | Select2 | Select3 | Select4 | Select5 | Select6 | Select7 |
|---|---|---|---|---|---|---|---|
| list3 | 36 | | | | | | |
| List3N1 | 23 | | | | R | | |
| | | | | | norm. H.R. | | |
| List3N2 | 22 | | R | | | | |
| | | | norm. H.R. | | | | |
| List3N3 | 23 | | Q | | | | |
| | | | chi | | | | |
| List3N4 | 15 | | | | Q | | |
| | | | | | chi | | |
| List3N5 | 23 | | | | T(10:40) | | |
| | | | | | chi | | |
| List3O1 | 10 | | | | | T(8:42) | |
| | | | | | | chi | |
| List3O2 | 15 | | | | | R | |
| | | | | | | H.R. | |
| List3P1 | 24 | | | | | R | |
| | | | | | | norm. H.R. | |
| List3P2 | 24 | | | | | | R |
| | | | | | | | norm. H.R. |
| List3P3 | 7 | | | R | | | |
| | | | | norm. H.R. | | | |
| List3P4 | 9 | | | | | Q | |
| | | | | | | chi | |
| List3P5 | 15 | | | | | | Q |
| | | | | | | | chi |
| List3P6 | 16 | | | Q | | | |
| | | | | chi | | | |
| List3P7 | 14 | | | | | T(10:40) | |
| | | | | | | chi | |
| List3P8 | 19 | | | | | | T(10:40) |
| | | | | | | | chi |
| List3P9 | 18 | | | T(10:40) | | | |
| | | | | chi | | | |
| List3P10 | 12 | R | | | | | |
| | | norm. H.R. | | | | | |
| List3P11 | 22 | Q | | | | | |
| | | chi | | | | | |
| List3P12 | 14 | T(10:40) | | | | | |
| | | chi | | | | | |
| List3qs | 18 | | | | | Q | |
| | | | | | | chi | |

TABLE 13

| | Raw | | | | Quarter | | | | Treshold | | | | Sensitivity | | Specif- | Fisher |
|---|---|---|---|---|---|---|---|---|---|---|---|---|---|---|---|---|
| | Stud- | | | LR > | Stud- | | | LR > | Stud- | | | LR > | Count > | | icity | Count < |
| | ies | Train | Val | 0.05 | ies | Train | Val | 0.05 | ies | Train | Val | 0.05 | 0.3 | AVG | AVG | 0.05 |
| list3 | 6 | 7 | 4 | 0 | 5 | 7 | 0 | 0 | 7 | 7 | 7 | 0 | 1 | 0.05 | 0.69 | 7 |
| list3_19 | 8 | 5 | 2 | 1 | 5 | 5 | 0 | 1 | 8 | 6 | 4 | 0 | 0 | 0.15 | 0.81 | 7 |
| List3N1 | 7 | 7 | 5 | 0 | 4 | 7 | 4 | 0 | 7 | 7 | 7 | 0 | 4 | 0.22 | 0.76 | 7 |
| List3N2 | 5 | 5 | 1 | 0 | 4 | 6 | 1 | 0 | 7 | 7 | 6 | 0 | 1 | 0.07 | 0.86 | 7 |
| List3N3 | 7 | 7 | 2 | 0 | 4 | 7 | 1 | 0 | 8 | 7 | 7 | 0 | 0 | 0.25 | 0.90 | 7 |
| List3N4 | 3 | 7 | 4 | 0 | 2 | 5 | 4 | 0 | 7 | 7 | 5 | 0 | 0 | 0.16 | 0.96 | 7 |
| List3N5 | 3 | 7 | 5 | 0 | 4 | 7 | 4 | 0 | 7 | 7 | 7 | 0 | 4 | 0.19 | 0.80 | 7 |
| List3O1 | 2 | 5 | 4 | 0 | 1 | 4 | 4 | 0 | 4 | 7 | 4 | 0 | 0 | 0.22 | 0.92 | 7 |
| List3O2 | 6 | 7 | 7 | 0 | 2 | 7 | 4 | 0 | 9 | 7 | 7 | 0 | 4 | 0.23 | 0.79 | 7 |
| List3O3 | 4 | 0 | 0 | 1 | 1 | 0 | 0 | 2 | 5 | 2 | 0 | 0 | 0 | 0.03 | 0.51 | 7 |
| List3O4 | 2 | 2 | 0 | 0 | 1 | 1 | 0 | 0 | 8 | 3 | 1 | 0 | 0 | 0.00 | 0.32 | 7 |
| List3O5 | 0 | 0 | 0 | 2 | 1 | 0 | 0 | 3 | 6 | 0 | 0 | 0 | 0 | 0.00 | 0.14 | 6 |
| List3P1 | 5 | 7 | 4 | 0 | 3 | 7 | 4 | 0 | 7 | 7 | 7 | 0 | 5 | 0.25 | 0.74 | 7 |
| List3P10 | 4 | 3 | 0 | 3 | 0 | 2 | 0 | 2 | 4 | 4 | 3 | 0 | 0 | 0.02 | 0.88 | 5 |
| List3P11 | 6 | 7 | 4 | 0 | 4 | 7 | 1 | 0 | 7 | 7 | 7 | 0 | 7 | 0.34 | 0.84 | 7 |
| List3P12 | 5 | 6 | 2 | 0 | 5 | 7 | 0 | 0 | 7 | 7 | 6 | 0 | 4 | 0.27 | 0.82 | 7 |
| List3P2 | 4 | 7 | 4 | 0 | 4 | 7 | 4 | 0 | 9 | 7 | 7 | 0 | 4 | 0.23 | 0.73 | 7 |
| List3P3 | 5 | 3 | 2 | 3 | 3 | 3 | 0 | 0 | 7 | 3 | 3 | 0 | 0 | 0.04 | 0.59 | 5 |
| List3P4 | 3 | 7 | 4 | 0 | 2 | 6 | 4 | 0 | 8 | 7 | 4 | 0 | 3 | 0.31 | 0.87 | 7 |
| List3P5 | 2 | 7 | 4 | 0 | 2 | 7 | 4 | 0 | 8 | 7 | 6 | 0 | 0 | 0.13 | 0.98 | 7 |
| List3P6 | 6 | 7 | 7 | 0 | 3 | 7 | 4 | 0 | 7 | 7 | 7 | 0 | 5 | 0.32 | 0.87 | 7 |
| List3P7 | 2 | 7 | 4 | 0 | 1 | 4 | 4 | 0 | 4 | 7 | 5 | 0 | 2 | 0.22 | 0.94 | 7 |
| List3P8 | 3 | 7 | 4 | 0 | 3 | 6 | 4 | 0 | 6 | 7 | 6 | 0 | 1 | 0.22 | 0.93 | 7 |
| List3P9 | 7 | 5 | 1 | 1 | 4 | 5 | 0 | 1 | 7 | 7 | 6 | 0 | 3 | 0.23 | 0.90 | 7 |

### REFERENCES

Ausubel et al. 1992 Current Protocols in Molecular Biology, Greene Publishing Associates (1992, and Supplements to 2002.
Beenken et al. 2001 Ann. Surg. 233(5):630-638.
Berx, G., Raspe, E., Christofori, G., Thiery, J. P., and Sleeman, J. P. 2007. Pre-EMTing metastasis?Recapitulation of morphogenetic processes in cancer. Clinical and Experimental Metastasis 24:587-597.
Bild, A. H., Yao, G., Chang, J. T., Wang, Q., Potti, A., Chasse, D., Joshi, M. B., Harpole, D., Lancaster, J. M., Berchuck, A., et al. 2006. Oncogenic pathway signatures in human cancers as a guide to targeted therapies. Nature 439:353-357.
Blanchard et al. Biosensors & Bioelectronics 11:687-690
Brummelkamp, T. R., Bernards, R., and Agami, R. 2002. Stable suppression of tumorigenicity by virus-mediated RNA interference. Cancer Cell 2:243-247.
Comijn, J., Berx, G., Vermassen, P., Verschueren, K., van Grunsven, L., Bruyneel, E., Mareel, M., Huylebroeck, D., and van Roy, F. 2001. The two-handed E box binding zinc finger protein SIP1 down-regulates E-cadherin and induces invasion. Mol Cell 7:1267-1278.
DeRisi et al. 1996 Nature Genetics 14:457-460.
Elloul, S., Elstrand, M. B., Nesland, J. M., Trope, C. G., Kvalheim, G., Goldberg, I., Reich, R., and Davidson, B. 2005. Snail, Slug, and Smad-interacting protein 1 as novel parameters of disease aggressiveness in metastatic ovarian and breast carcinoma. Cancer 103:1631-1643.
Ferguson et al. 1996 Nature Biotech. 14:1681-1684.
Fodor et al. 1991 Science 251:767-773
Harlow and Lane, 1990 Antibodies: A Laboratory Manual, Cold Spring Harbor Laboratory Press, Cold Spring Harbor, N.Y.
Held et al. 1996 Genome Research 6:986-994.
Isaacs et al. 2001 Sernin. Oncol. 28(1):53-67.
Jemal, A., Siegel, R., Ward, E., Murray, T., Xu, J., and Thun, M. J. 2007. Cancer statistics, 2007. CA Cancer J Clin 57:43-66.
Lanigan, F., O'Connor, D., Martin, F., and Gallagher, W. M. 2007. Molecular links between mammary gland development and breast cancer. Cell Mol Life Sci 64:3159-3184.
Lockhart et al. 1996 Nature Biotechnology 14:1675
McCall, M. N., Bolstad, B. M., and Irizarry, R. A. 2010. Frozen robust multiarray analysis (fRMA). Biostatistics 11:242-253.
Miki et al. 1994 Science, 266:66-71.
Neve, R. M., Chin, K., Fridlyand, J., Yeh, J., Baehner, F. L., Fevr, T., Clark, L., Bayani, N., Coppe, J. P., Tong, F., et al. 2006. A collection of breast cancer cell lines for the study of functionally distinct cancer subtypes. Cancer Cell 10:515-527.
Pawitan, Y., Bjohle, J., Amler, L., Borg, A. L., Egyhazi, S., Hall, P., Han, X., Holmberg, L., Huang, F., Klaar, S., et al. 2005. Gene expression profiling spares early breast cancer patients from adjuvant therapy: derived and validated in two population-based cohorts. Breast Cancer Res 7:R953-964.
Pease et al. 1994 Proc. Natl. Acad. Sci. U.S.A. 91:5022-5026
Perou, C. M., Sorlie, T., Eisen, M. B., van de Rijn, M., Jeffrey, S. S., Rees, C. A., Pollack, J. R., Ross, D. T., Johnsen, H., Akslen, L. A., et al. 2000. Molecular portraits of human breast tumours. Nature 406:747-752.
Rodenhiser, D. I., Andrews, J., Kennette, W., Sadikovic, B., Mendlowitz, A., Tuck, A. B., and Chambers, A. F. 2008. Epigenetic mapping and functional analysis in a breast cancer metastasis model using whole-genome promoter tiling microarrays. Breast Cancer Res 10:R62.
Sambrook et al. 1989 Molecular Cloning: A Laboratory Manual, 2d ed., Cold Spring Harbor Laboratory Press, Cold Spring Harbor, N.Y.
Sarrio, D., Rodriguez-Pinilla, S. M., Hardisson, D., Cano, A., Moreno-Bueno, G., and Palacios, J. 2008. Epithelial-mesenchymal transition in breast cancer relates to the basal-like phenotype. Cancer Res 68:989-997.
Schena et al. 1995b Proc. Natl. Acad. ScL U.S.A. 93:10539-11286.
Schena et al. 1993 Proc. Natl. Acad. ScL U.S.A. 93:10614.
Schena et al. 1995a Science 270:467-470.
Schena et al. 1996 Genome Res. 6:639-645.
Shalon et al. 1996 Genome Res. 5:639-645.
Shimono, Y., Zabala, M., Cho, R. W., Lobo, N., Dalerba, P., Qian, D., Diehn, M., Liu, H., Panula, S. P., Chiao, E., et al. 2009. Down-regulation of miRNA-200c links breast cancer stem cells with normal stem cells. Cell 138:592-603.
Sorlie, T., Perou, C. M., Tibshirani, R., Aas, T., Geisler, S., Johnsen, H., Hastie, T., Eisen, M. B., van de Rijn, M., Jeffrey, S. S., et al. 2001. Gene expression patterns of breast carcinomas distinguish tumor subclasses with clinical implications. Proc Natl Acad Sci USA 98:10869-10874.
Sorlie, T., Wang, Y., Xiao, C., Johnsen, H., Naume, B., Samaha, R. R., and Borresen-Dale, A. L. 2006. Distinct molecular mechanisms underlying clinically relevant subtypes of breast cancer: gene expression analyses across three different platforms. BMC Genomics 7:127.
Sotiriou, C., Wirapati, P., Loi, S., Harris, A., Fox, S., Smeds, J., Nordgren, H., Farmer, P., Praz, V., Haibe-Kains, B., et al. 2006. Gene expression profiling in breast cancer: understanding the molecular basis of histologic grade to improve prognosis. J Natl Cancer Inst 98:262-272.
Thiery, J. P., Acloque, H., Huang, R. Y., and Nieto, M. A. 2009. Epithelial-mesenchymal transitions in development and disease. Cell 139:871-890.
Ui-Tei, K., Naito, Y., Takahashi, F., Haraguchi, T., Ohki-Hamazaki, H., Juni, A., Ueda, R., and Saigo, K. 2004. Guidelines for the selection of highly effective siRNA sequences for mammalian and chick RNA interference. Nucleic Acids Res 32:936-948.
Vandesompele J, De Preter K, Pattyn F, Poppe B, Van Roy N, De Paepe A, Speleman F. 2002. Accurate normalization of real-time quantitative RT-PCR data bygeometric averaging of multiple internal control genes. Genome Biol. 2002 3(7):RESEARCH0034.
Vandewalle, C., Comijn, J., De Craene, B., Vermassen, P., Bruyneel, E., Andersen, H., Tulchinsky, E., Van Roy, F., and Berx, G. 2005. SIP1/ZEB2 induces EMT by repressing genes of different epithelial cell-cell junctions. Nucleic acids research 33:6566-6578.
Van't Veer et al. 2002 Nature 415(6871):530-536.
Wiznerowicz, M., and Trono, D. 2003. Conditional suppression of cellular genes: lentivirus vector-mediated drug-inducible RNA interference. J Virol 77:8957-8961.

1. A method of prognosing an individual suffering from or suspected as suffering from breast cancer, the method comprising the steps of:
(i) providing a sample from the individual comprising breast cancer cells or suspected to comprise breast cancer cells;
(ii) establishing a gene expression profile by quantifying in said sample the expression level of a plurality of genes comprising any combination of at least 8 genes from Table 1;
(iii) comparing said gene expression profile with a reference gene expression profile; and
(iv) classifying the individual as having a good prognosis or a poor prognosis according to the comparison in step (iii).

2. The method of claim 1 wherein said reference gene expression profile is established by quantifying the differential expression level of the corresponding at least 8 genes as quantified in at least two reference samples that differentially express ZEB2.

3. The method of claim 2 wherein a first reference sample endogenously expresses ZEB2 and wherein a second reference sample only differs from the first in that the expression of ZEB2 is knocked-down.

4. The method of claim 2, wherein said reference sample is a reference cell line.

5. The method of claim 4, wherein said reference cell line is a basal-like breast cancer cell line.

6. The method of claim 1, wherein the expression level of the at least 8 genes is quantified by measuring the level of transcription.

7. The method of claim 1, wherein an increasing correlation coefficient between the gene expression profile and the reference gene expression profile indicates a poor prognosis for breast cancer in the individual, and wherein a decreasing correlation coefficient between the gene expression profile and the reference gene expression profile indicates a good prognosis for breast cancer in the individual.

8. The method of claim 1, wherein the sensitivity and/or specificity of the method is at least 80%.

9. A method for monitoring a change in the prognosis of an individual suffering from or suspected to suffer from breast cancer, the method comprising the steps of:
(i) applying the method of claim 1 to the individual at one or more successive time points, whereby the prognosis of breast cancer in the individual is determined at said successive time points;
(ii) comparing the prognosis of breast cancer in the individual at said successive time points as determined in (i); and
(iii) finding the presence or absence of a change between the prognosis of breast cancer in the individual at said successive time points as determined in step (i).

10. The method according to claim 9, wherein said change in prognosis of breast cancer in the individual is monitored in the course of a medical treatment of the individual.

11. A kit for prognosing an individual suffering from or suspected of suffering from breast cancer, the kit comprising:
(i) means for establishing a gene expression profile by quantifying, in a sample of the individual comprising breast cancer cells or suspected to comprise breast cancer cells, the expression level of a plurality of genes comprising any combination of at least 8 genes from Table 1;
(ii) means for comparing the gene expression profile thereby established with a reference gene expression profile; and
(iii) means for classifying the individual as having a good or poor prognosis per said comparison.

12. An oligonucleotide array or microarray comprising a plurality of probes complementary and hybridizable to nucleotide sequences of any combination of at least 8 genes from Table 1, wherein said plurality of probes is at least 50% of the probes on said array or microarray.

13. A gene expression profile indicative of a good prognosis or a poor prognosis of an individual suffering from or suspected of suffering from breast cancer, said gene expression profile comprising a quantified expression level of a plurality of genes comprising any combination of at least 8 genes from Table 1.

14. A reference gene expression profile established by quantifying differential expression level of the corresponding at least 8 genes as quantified in at least two reference samples that differentially express ZEB2.

15. (canceled)

16. The reference gene expression profile of claim 14, wherein a first reference sample endogenously expresses ZEB2 and wherein a second reference sample only differs from the first in that the expression of ZEB2 is knocked-down.

17. The reference gene expression profile of claim 14, wherein the reference sample is a breast cell line or a breast cancer cell line.

18. The reference gene expression profile of claim 17, wherein the reference cell line is a MDAMB231 cell line.

19. The method according to claim 4, wherein the reference sample is a breast cell line or a breast cancer cell line.

20. The method according to claim 19, wherein the reference cell line is a MDAMB231 cell line.
